SEQUENCE LISTING (35 sequences)

SEQ ID NO: 1 - DNA, 6946 bp, Zea mays L.
gcaaatccatatgctcagctcccgcctcctcccatccccggaccccggaccccggccatgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaaggtgagctgccgcctgcccccccgcaccccaagccgccgcgctgtccctgtctctgtctctcctactagtagtagtagctggtggtgattccgagcgcgtctttggtctggtgcatcgaaccacttgtgcttggtgcatttcgaggggattcggtgtaattccgtgcaaattggggatttctctcctgttgctttccgaggtttaggtgtttcgattgggacgcgattggagccgttcattttaggacatttccggtgccttttgggaggcgtttagctcaacgagtagctcactcacatttctagctgtttggccgcttcatttctcccaagctttcgttgtttgccggtggttctgagctgcgggatcttgacgttggccagagaggtggtttcgacattcaggcatctcggatgacctcttagtttggcactacagctctattatttcgggaacgacgtgttgctcagtgcgcacctcattcatggaagtggcaaggtcgcttgtctgcagaacggggaaggtgcttttcatctggctattcatggaaaacgacttgttcagttgccctactaataatttcaataagattgcctgcctccttgaatggttggggcttggaaggttcctgtcgaagaaaaagtcaggaaagataacaattgcgcacttgcagtggacaacgcttccctgtcttctatgctataggtggacagcatttttctaggtataattaatttgaccttcaaacatatgtatactaaccaacgcggttttgattccatcaaatgttttggactctctctgctgaactgtcaaagttacttcatggggcaaaatgtcaaattttctggaaccttccgtagtatattttggaaatgagtgtttattgtgtcattggaaataccgttcatgtgtctgtgacagaatgtgtcactagaaagctgaattggtgttgtccttgtcaaaaaggcactaaacacgagtctgaaaattaggcctgttcttggtaagggaaggaatctgagcatcaatgctgataggaatagactctgtctgtcaatattgttaacttgtttatagggcttcgagttttcaacttttgaggcagataagtaggatacctcttttgatcatgatatataacatattcttatatacctcaagccttgcactgttaagttaatgtggcatcctttctagagatcatgacctcaagttgcatatggatgccaataatatcgacaccaagtgaacatcagtgtctgtggaatatgccgaaagcagccaacgtgccattactgaattttcatatgattattatattctgtttagatttatttacgtcggaacacagtgagatggtaacgtaatgaatcaaaataggctataaacatgcaattcaacatatcattatcatgcccaagtgttttgtcattctatctttattcgtccaagaaggacaagcctggtgcattgttgagggaaccagttcttctgcagtacttctagggaggtaaaaattcaacaccgttggatgcagatctatcgaacccagggactttgtgcttccagtgaaaagttatatggacccataggccagaggatgtgagagttttacctctctggaagttatatgcgctagcattagtgtggtcatcaatgggatcaaagatgagctccacctttggtgtagagctggagctaggggactctagcatcctggcgctccaatcttccatccagtgaactctgttttttgggtctagtaggtcaagggtccagttattttttctttctgctgtaaagtctctagttaaggtgtgagttttgtatggtgttttttcgaggtttccccaaacctcaccttttttccttcttaatataatgatatgcagctttcctgcgtattcgagaaaagaaagttttatctctctggaagttaactgcagaggaacttgttacattgttgagagttgtctcaccgagtcaccaggtcgctggttcaaagcagtctctccacatttatgtggaaggcttgcctcggtttatcccttcccaagactctacttgtgggagactctggcattgggtctgtcctatgccgttgaagcgctaggttcgtttatcccttccctatacccacttgtcagagcctccaacactgagtctgccctaagcttccaagttccaacactgggtctgccctaggccgtcgaagcgttatatgattgccatgtactgttatgctttgttgccttcacatattttccgttcgaaatcatctccttgttgccttcacatattgccttgttgctttcacatattttctgttccacgtcatacttagaagttagaacacgtgatttatgccaattaagattattattttatataacagatgacccgccctgttgctgtggaggatgtgaaaagagaagtgaagattcttaaagcacttaaaggacatcagaatattgttcacttctacaatgcatttgaggatgattcatacgtgtacattgtgatggagtaagtaggcccatacacctgttcctgctaatagagcatatcgattttgctatgacttttttccctaaagttttaacatgaacaatatctatcctgtttacagaatcctagacactaaaatgtcatttctaattatcaattattctatagctaaaccagatgcaatcctgatttatttttcttaacgtatggatatattggacttttctttcaaacctgcattttgaatttgattacagggaactataacactaattcagaactctatcatgtttaacatttttcttgcattgttctatgtttgtcaacttgacgcacttcttagataatataacatcatcttccacagtcaccattagttaggaccttggaccttcatggttccgaaatttagctaagaatggtatacatggtcatgtgatttcaaatagatgttcctatatgccagaaccaactcataagtcataagttttaccttgtgtttttgcaggctatgtgagggcggtgaactattagatcggattttggcaaagtaagtagataagatccccatctctttgtttcccgtacctcattcttcgccattaaatttatagatttttgtgctgtaaaatcagattgctttatgttgtttgtctgctttgtttgatttctagttgctcgttcaagatcctttacttaatggtgtgcgtgttttgacagaaagaatagccgctatagtgagaaagatgctgcagtggtagtccgccaaatgctcaaagtagctgctgaatgccatctgcgtgggttagttcaccgagatatgaagcctgaggtagaaatcaaatacttcaatctctttgcacacagtaagcatttggtgatatttcactacttcctcaggtcatgtaagactgtacctattttccttcccagaacttccttttcaaatcgaacaaggaggattcaccactaaaggcgacagattttggtttgtcagatttcattaagccaggtatctacttggggccatctgaatctgtcgggaatctgataggggcaagtctgcagtttagctgaccattttgttgtctaatgcatgctttagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggtaagtggatggattttgcataccatgtgcttacatgtaaaatatgcttggttagagtgctgtaccagggatcagcgttttcagcgtgctgatactgttttgtacaatgtgtttctactttctacgtcatatagcagtgtttctttgttaactatttcagtgtcaaactatttgtcgtgtcacaactcagcagtataattttactattttgaacactgtaaacctgcctggtcaggttatccttcagtaatttctctactagctaccagaaacccactttatgcaggtgttcagtttaataacacccaccatctttcagatttctaatgttcagtgttagacagacttcattaagatgcaccttaagatgattgtaagtagtaaaagtgctttgcacttttgttaacttttgagtctgaagatgacttgtggtacctatgacctcaagaaaccaaggcattgccattggaatagctaattcgaatgagcttcagatatggctatctgttttagttttggacatctgactcaactttataggataatactatattagcaatctttgaggtcattgtctcagccaaaataagttgcggtctcttttttactgtcctaagcagcaatatggtttccattttcattataccagcaacttccacctttttcttgctatttaaatatctttatgcattttatcagcaaggacatgatacgatcgtatatgtgatattctacatcttttcacttctcataattaggttctaaggaacaagcctgattttcgtaagaggccttggtcaagcatcagcccaggtgctaaagattttgttaaaaggttactagtgaagaatccaagggccaggctaacagctgctcaagctctctgtaagttttggtatttttcattaatttactagcctagtcatgatgatcagattcaccttctctatgtgagaacagagaacacatatacatctggcagtatgcctttcaatcagttatgacaatgtaaatatgcaaagaccgatgttttttctatcctgcaccattttagaacattaatggggaaaaaccacaatatattaggaaaaatgtttaattatgtcctggtcacttgaaatgaacatataccactgaggttttctagttctcatgcgttcttataatgatctaataagtcagtggaggtttgctgcccaccacccctacatttgtattgtgaattactatcatctttactgatcctgattgttcttgatatgttaagcacatccgtgggtaagagaaggaggggaagcatccgatatccccgtcgacatatctgtgttatcaaacatgcgtcagtttgtcaagtacagccgtttcaagcaattcgcgcttcgggtaattacagtgattacaaaaaacaacactgcatcgtttattttttcctcacaatatttcctcgtggcatggtcaggctctggcgagcacccttaacgaggaagagctatcagatctgaaggatcagtttgatgcaattgatatcgataaaagtggatcgattagtatcgaggaaatgcgtcatgtaggttctgttagtgtttgctgatgaaaatgccttagatcctgaactactctgcggtgctgattaatctgtgcatgtttcggtaggcccttgcaaaggatcttccctggagattgaagggtccccgtgtgctggagattattcaagcagtaagtttgagccttcttctggatccagccctttctttgttaccccccttgtttccaagaaaatagctggccttgttctgagggtataaccaaaactgcatcttattttgtggtagattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatggtaattttctactcctgtcttgtttccatgttgcttcaccaacgaatgcacagttcacataacccttattatcatcactgcttcccatgaataactagctggctcgaccatcatgagattcagtacttgcgccctgtgcacttggttttggtcccgcttgttagaatgaagtaatttatcaatggaagcgctgtaatattttaatcagcgtttagatttgataaagataaaacatgttcattgtttgtgccaagaaatccacttacacagatactgagagttgcaccgtagataacgctaatcggcagtatcctaatcgagattttctttcaaggtgcagcaccctgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctgtgaattccggctcggccactagggaggagcaagcttaggaagttgccatacaatagccatgtgttctttgggttcttcagagtgccatgtgatgtttctggtttttagcatccaggttatgtgtgcagtgcagccccgagtgagtttcgaagtaaatattcagtgctttctttttctttccggaagagtgagaggtggaggtcaaaatggtaggcaagactcgccttcttctttcctttacactgtacagtgatactgaaatatgtacgatttttattataactgttcgtcgcaataaagttatttggagaagtgagga

SEQ ID NO: 2 - protein, 520 aa, Zea mays L.
MGACFSSASAAPAGAAVDERRPSKEGDGKKRRRAAGASPDAAAPVRVEFGYERDFEARYEVGRLLGHGQFGYTFAATDRGSGDRVAVKRIDKAKMTRPVAVEDVKREVKILKALKGHQNIVHFYNAFEDDSYVYIVMELCEGGELLDRILAKKNSRYSEKDAAVVVRQMLKVAAECHLRGLVHRDMKPENFLFKSNKEDSPLKATDFGLSDFIKPGKKFHDIVGSAYYVAPEVLKRRSGPESDVWSIGVITYILLCGRRPFWDKTEDGIFKEVLRNKPDFRKRPWSSISPGAKDFVKRLLVKNPRARLTAAQALSHPWVREGGEASDIPVDISVLSNMRQFVKYSRFKQFALRALASTLNEEELSDLKDQFDAIDIDKSGSISIEEMRHALAKDLPWRLKGPRVLEIIQAIDSNTDGLVDFKEFVAATLHIHQMAELDSERWGIRCQAAFSKFDLDGDGYITPEELRMHPGLKGSIEPLLEEADIDKDGKISLSEFRKLLRTASMSNVPSPRGPPNPQAL

SEQ ID NO: 3 - DNA, 1934 bp, Zea mays L.
aaatccatatgctcagctcccgcctcctcccatccccggaccccggaccccggccatgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaagatgacccgccctgttgctgtggaggatgtgaaaagagaagtgaagattcttaaagcacttaaaggacatcagaatattgttcacttctacaatgcatttgaggatgattcatacgtgtacattgtgatggagctatgtgagggcggtgaactattagatcggattttggcaaaaaagaatagccgctatagtgagaaagatgctgcagtggtagtccgccaaatgctcaaagtagctgctgaatgccatctgcgtgggttagttcaccgagatatgaagcctgagaacttccttttcaaatcgaacaaggaggattcaccactaaaggcgacagattttggtttgtcagatttcattaagccagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggttctaaggaacaagcctgattttcgtaagaggccttggtcaagcatcagcccaggtgctaaagattttgttaaaaggttactagtgaagaatccaagggccaggctaacagctgctcaagctctctcacatccgtgggtaagagaaggaggggaagcatccgatatccccgtcgacatatctgtgttatcaaacatgcgtcagtttgtcaagtacagccgtttcaagcaattcgcgcttcgggctctggcgagcacccttaacgaggaagagctatcagatctgaaggatcagtttgatgcaattgatatcgataaaagtggatcgattagtatcgaggaaatgcgtcatgcccttgcaaaggatcttccctggagattgaagggtccccgtgtgctggagattattcaagcaattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatgcaccctgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctgtgaattccggctcggccactagggaggagcaagcttaggaagttgccatacaatagccatgtgttctttgggttcttcagagtgccatgtgatgtttctggtttttagcatccaggttatgtgtgcagtgcagccccgagtgagtttcgaagtaaatattcagtgctttctttttctttccggaagagtgagaggtggaggtcaaaatggtaggcaagactcgccttcttctttcctttacactgtacagtgatactgaaatatgtacgatttttattataactgttcgtcgcaataaagttatttggagaagtgagga

SEQ ID NO: 4 - protein, 522 aa, Zea mays L.
MGACFSSASAAPAGAAVDERRPSKEGDGKKRRRAAGASPDAAAPVRVEFGYERDFEARYEVGRLLGHGQFGYTFAATDRGSGDRVAVKRIDKAKMTRPVAVEDVKREVKILKALKGHQNIVHFYNAFEDDSYVYIVMELCEGGELLDRILAKKNSRYSEKDAAVVVRQMLKVAAECHLRGLVHRDMKPENFLFKSNKEDSPLKATDFGLSDFIKPGKKFHDIVGSAYYVAPEVLKRRSGPESDVWSIGVITYILLCGRRPFWDKTEDGIFKEVLRNKPDFRKRPWSSISPGAKDFVKRLLVKNPRARLTAAQALSHPWVREGGEASDIPVDISVLSNMRQFVKYSRFKQFALRALASTLNEEELSDLKDQFDAIDIDKSGSISIEEMRHALAKDLPWRLKGPRVLEIIQAIDSNTDGLVDFKEFVAATLHIHQMAELDSERWGIRCQAAFSKFDLDGDGYITPEELRMVQHPGLKGSIEPLLEEADIDKDGKISLSEFRKLLRTASMSNVPSPRGPPNPQAL

SEQ ID NO: 5 - DNA, 1940 bp, Zea mays L.
aaatccatatgctcagctcccgcctcctcccatccccggaccccggaccccggccatgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaagatgacccgccctgttgctgtggaggatgtgaaaagagaagtgaagattcttaaagcacttaaaggacatcagaatattgttcacttctacaatgcatttgaggatgattcatacgtgtacattgtgatggagctatgtgagggcggtgaactattagatcggattttggcaaaaaagaatagccgctatagtgagaaagatgctgcagtggtagtccgccaaatgctcaaagtagctgctgaatgccatctgcgtgggttagttcaccgagatatgaagcctgagaacttccttttcaaatcgaacaaggaggattcaccactaaaggcgacagattttggtttgtcagatttcattaagccagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggttctaaggaacaagcctgattttcgtaagaggccttggtcaagcatcagcccaggtgctaaagattttgttaaaaggttactagtgaagaatccaagggccaggctaacagctgctcaagctctctcacatccgtgggtaagagaaggaggggaagcatccgatatccccgtcgacatatctgtgttatcaaacatgcgtcagtttgtcaagtacagccgtttcaagcaattcgcgcttcgggctctggcgagcacccttaacgaggaagagctatcagatctgaaggatcagtttgatgcaattgatatcgataaaagtggatcgattagtatcgaggaaatgcgtcatgcccttgcaaaggatcttccctggagattgaagggtccccgtgtgctggagattattcaagcaattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatggtgcagcaccctgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctgtgaattccggctcggccactagggaggagcaagcttaggaagttgccatacaatagccatgtgttctttgggttcttcagagtgccatgtgatgtttctggtttttagcatccaggttatgtgtgcagtgcagccccgagtgagtttcgaagtaaatattcagtgctttctttttctttccggaagagtgagaggtggaggtcaaaatggtaggcaagactcgccttcttctttcctttacactgtacagtgatactgaaatatgtacgatttttattataactgttcgtcgcaataaagttatttggagaagtgagga

SEQ ID NO: 6 - DNA, 6946 bp, Zea mays L.
gcaaatccatatgctcagctcccgcctcctcccatccccggaccccggaccccggccatgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaaggtgagctgccgcctgcccccccgcaccccaagccgccgcgctgtccctgtctctgtctctcctactagtagtagtagctggtggtgattccgagcgcgtctttggtctggtgcatcgaaccacttgtgcttggtgcatttcgaggggattcggtgtaattccgtgcaaattagggatttctctcctgttgctttccgaggtttaggtgtttcgattgggacgcgattggagccgttcattttaggacatttccggtgccttttgggaggcgtttagctcaacgagtagctcactcacatttctagctgtttggccgcttcatttctcccaagctttcgttgtttgccggtggttctgagctgcgggatcttgacgttggccagagaggtggtttcgacattcaggcatctcggatgacctcttagtttggcactacagctctattatttcgggaacgacgtgttgctcagtgcgcacctcattcatggaagtggcaaggtcgcttgtctgcagaacggggaaggtgcttttcatctggctattcatggaaaacgacttgttcagttgccctactaataatttcaataagattgcctgcctccttgaatggttggggcttggaaggttcctgtcgaagaaaaagtcaggaaagataacaattgcgcacttgcagtggacaacgcttccctgtcttctatgctataggtggacagcatttttctaggtataattaatttgaccttcaaacatatgtatactaaccaacgcggttttgattccatcaaatgttttggactctctctgctgaactgtcaaagttacttcatggggcaaaatgtcaaattttctggaaccttccgtagtatattttggaaatgagtgtttattgtgtcattggaaataccgttcatgtgtctgtgacagaatgtgtcactagaaagctgaattggtgttgtccttgtcaaaaaggcactaaacacgagtctgaaaattaggcctgttcttggtaagggaaggaatctgagcatcaatgctgataggaatagactctgtctgtcaatattgttaacttgtttatagggcttcgagttttcaacttttgaggcagataagtaggatacctcttttgatcatgatatataacatattcttatatacctcaagccttgcactgttaagttaatgtggcatcctttctagagatcatgacctcaagttgcatatggatgccaataatatcgacaccaagtgaacatcagtgtctgtggaatatgccgaaagcagccaacgtgccattactgaattttcatatgattattatattctgtttagatttatttacgtcggaacacagtgagatggtaacgtaatgaatcaaaataggctataaacatgcaattcaacatatcattatcatgcccaagtgttttgtcattctatctttattcgtccaagaaggacaagcctggtgcattgttgagggaaccagttcttctgcagtacttctagggaggtaaaaattcaacaccgttggatgcagatctatcgaacccagggactttgtgcttccagtgaaaagttatatggacccataggccagaggatgtgagagttttacctctctggaagttatatgcgctagcattagtgtggtcatcaatgggatcaaagatgagctccacctttggtgtagagctggagctaggggactctagcatcctggcgctccaatcttccatccagtgaactctgttttttgggtctagtaggtcaagggtccagttattttttctttctgctgtaaagtctctagttaaggtgtgagttttgtatggtgttttttcgaggtttccccaaacctcaccttttttccttcttaatataatgatatgcagctttcctgcgtattcgagaaaagaaagttttatctctctggaagttaactgcagaggaacttgttacattgttgagagttgtctcaccgagtcaccaggtcgctggttcaaagcagtctctccacatttatggggaaggcttgcctcggtttatcccttcccaagactctacttgtgggagactctggcattgggtctgtcctatgccgttgaagcgctaggttcgtttatcccttccctatacccacttgtcagagcctccaacactgagtctgccctaagcttccaagttccaacactgggtctgccctaggccgtcgaagcgttatatgattgccatgtactgttatgctttgttgccttcacatattttccgttcgaaatcatctccttgttgccttcacatattgccttgttgctttcacatattttctgttccacgtcatacttagaagttagaacacgtgatttatgccaattaagattattattttatataacagatgacccgccctgttgctgtggaggatgtgaaaagagaagtgaagattcttaaagcacttaaaggacatcagaatattgttcacttctacaatgcatttgaggatgattcatacgtgtacattgtgatggagtaagtaggcccatacacctgttcctgctaatagagcatatcgattttgctatgacttttttccctaaagttttaacatgaacaatatctatcctgtttacagaatcctagacactaaaatgtcatttctaattatcaattattctatagctaaaccagatgcaatcctgatttatttttcttaacgtatggatatattggacttttctttcaaacctgcattttgaatttgattacagggaactataacactaattcagaactctatcatgtttaacatttttcttgcattgttctatgtttgtcaacttgacgcacttcttagataatataacatcatcttccacagtcaccattagttaggaccttggaccttcatggttccgaaatttagctaagaatggtatacatggtcatgtgatttcaaatagatgttcctatatgccagaaccaactcatgagtcataagttttaccttgtgtttttgcaggctatgtgagggcggtgaactattagatcggattttggcaaagtaagtagataagatccccatctctttgtttcccgtacctcattcttcgccattaaatttatagatttttgtgctgtaaaatcagattgctttatgttgtttgtctgctttgtttgatttctagttgctcgttcaagatcctttacttaatggtgtgcgtgttttgacagaaagaatagccgctatagtgagaaagatgctgcagtggtagtccgccaaatgctcaaagtagctgctgaatgccatctgcgtgggttagttcaccgagatatgaagcctgaggtagaaatcaaatacttcaatctctttgcacacagtaagcatttggtgatatttcactacttcctcaggtcatgtaagactgtacctattttccttcccagaacttccttttcaaatcgaacaaggaggattcaccactaaaggcgacagattttggtttgtcagatttcattaagccaggtatctacttggggccatctgaatctgtcgggaatctgataggggcaagtctgcagtttagctgaccattttgttgtctaatgcatgctttagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggtaagtggatggattttgcataccatgtgcttacatgtaaaatatgcttggttagagtgctgtaccagggatcagcgttttcagcgtgctgatactgttttgtacaatgtgtttctactttctacgtcatatagcagtgtttctttgttaactatttcagtgtcaaactatttgtcgtgtcacaactcagcagtataattttactattttgaacactgtaaacctgcctggtcaggttatccttcagtaatttctctactagctaccagaaacccactttatgcaggtgttcagtttaataacacccaccatctttcagatttctaatgttcagtgttagacagacttcattaagatgcaccttaagatgattgtaagtagtaaaagtgctttgcacttttgttaacttttgagtctgaagatgacttgtggtacctatgacctcaagaaaccaaggcattgccattggaatagctaattcgaatgagcttcagatatggctatctgttttagttttggacatctgactcaactttataggataatactatattagcaatctttgaggtcattgtctcagccaaaataagttgcggtctcttttttactgtcctaagcagcaatatggtttccattttcattataccagcaacttccacctttttcttgctatttaaatatctttatgcattttatcagcaaggacatgatacgatcgtatatgtgatattctacatcttttcacttctcataattaggttctaaggaacaagcctgattttcgtaagaggccttggtcaagcatcagcccaggtgctaaagattttgttaaaaggttactagtgaagaatccaagggccaggctaacagctgctcaagctctctgtaagttttggtatttttcattaatttactagcctagtcatgatgatcagattcaccttctctatgtgagaacagagaacacatatacatctggcagtatgcctttcaatcagttatgacaatgtaaatatgcaaagaccgatgttttttctatcctgcaccattttagaacattaatggggaaaaaccacaatatattaggaaaaatgtttaattatgtcctggtcacttgaaatgaacatataccactgaggttttctagttctcatgcgttcttataatgatctaataagtcagtggaggtttgctgcccaccacccctacatttgtattgtgaattactatcatctttactgatcctgattgttcttgatatgttaagcacatccgtgggtaagagaaggaggggaagcatccgatatccccgtcgacatatctgtgttatcaaacatgcgtcagtttgtcaagtacagccgtttcaagcaattcgcgcttcgggtaattacagtgattacaaaaaacaacactgcatcgtttattttttcctcacaatatttcctcgtggcatggtcaggctctggcgagcacccttaacgaggaagagctatcagatctgaaggatcagtttgatgcaattgatatcgataaaagtggatcgattagtatcgaggaaatgcgtcatgtaggttctgttagtgtttgctgatgaaaatgccttagatcctgaactactctgcggtgctgattaatctgtgcatgtttcggtaggcccttgcaaaggatcttccctggagattgaagggtccccgtgtgctggagattattcaagcagtaagtttgagccttcttctggatccagccctttctttgttaccccccttgtttccaagaaaatagctggccttgttctgagggtataaccaaaactgcatcttattttgtggtagattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatggtaattttctactcctgtcttgtttccatgttgcttcaccaacgaatgcacagttcacataacccttattatcatcactgcttcccatgaataactagctggctcgaccatcatgagattcagtacttgcgccctgtgcacttggttttggtcccgcttgttagaatgaagtaatttatcaatggaagcgctgtaatattttaatcagcgtttagatttgataaagataaaacatgttcattgtttgtgccaagaaatccacttacacagatactgagagttgcaccgtagataacgctaatcggcagtatcctaatcgagattttctttcaaggtgcagcacactgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctgtgaattccggctcggccactagggaggagcaagcttaggaagttgccatacaatagccatgtgttctttgggttcttcagagtgccatgtgatgtttctggtttttagcatccaggttatgtgtgcagtgcagccccgagtgagtttcgaagtaaatattcagtgctttctttttctttccggaagagtgagaggtggaggtcaaaatggtaggcaagactcgccttcttctttcctttacactgtacagtgatactgaaatatgtacgatttttattataactgttcgtcgcaataaagttatttggagaagtgagga

SEQ ID NO: 7 - protein, 520 aa, Zea mays L.
MGACFSSASAAPAGAAVDERRPSKEGDGKKRRRAAGASPDAAAPVRVEFGYERDFEARYEVGRLLGHGQFGYTFAATDRGSGDRVAVKRIDKAKMTRPVAVEDVKREVKILKALKGHQNIVHFYNAFEDDSYVYIVMELCEGGELLDRILAKKNSRYSEKDAAVVVRQMLKVAAECHLRGLVHRDMKPENFLFKSNKEDSPLKATDFGLSDFIKPGKKFHDIVGSAYYVAPEVLKRRSGPESDVWSIGVITYILLCGRRPFWDKTEDGIFKEVLRNKPDFRKRPWSSISPGAKDFVKRLLVKNPRARLTAAQALSHPWVREGGEASDIPVDISVLSNMRQFVKYSRFKQFALRALASTLNEEELSDLKDQFDAIDIDKSGSISIEEMRHALAKDLPWRLKGPRVLEIIQAIDSNTDGLVDFKEFVAATLHIHQMAELDSERWGIRCQAAFSKFDLDGDGYITPEELRMHTGLKGSIEPLLEEADIDKDGKISLSEFRKLLRTASMSNVPSPRGPPNPQAL

SEQ ID NO: 8 - DNA, 1936 bp, Zea mays L.
aaatccatatgctcagctcccgcctcctcccatccccggaccccggaccccggccatgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaagatgacccgccctgttgctgtggaggatgtgaaaagagaagtgaagattcttaaagcacttaaaggacatcagaatattgttcacttctacaatgcatttgaggatgattcatacgtgtacattgtgatggagctatgtgagggcggtgaactattagatcggattttggcaaaaaagaatagccgctatagtgagaaagatgctgcagtggtagtccgccaaatgctcaaagtagctgctgaatgccatctgcgtgggttagttcaccgagatatgaagcctgagaacttccttttcaaatcgaacaaggaggattcaccactaaaggcgacagattttggtttgtcagatttcattaagccagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggttctaaggaacaagcctgattttcgtaagaggccttggtcaagcatcagcccaggtgctaaagattttgttaaaaggttactagtgaagaatccaagggccaggctaacagctgctcaagctctctcacatccgtgggtaagagaaggaggggaagcatccgatatccccgtcgacatatctgtgttatcaaacatgcgtcagtttgtcaagtacagccgtttcaagcaattcgcgcttcgggctctggcgagcacccttaacgaggaagagctatcagatctgaaggatcagtttgatgcaattgatatcgataaaagtggatcgattagtatcgaggaaatgcgtcatgcccttgcaaaggatcttccctggagattgaagggtccccgtgtgctggagattattcaagcaattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatgcacactgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctgtgaattccggctcggccactagggaggagcaagcttaggaagttgccatacaatagccatgtgttctttgggttcttcagagtgccatgtgatgtttctggtttttagcatccaggttatgtgtgcagtgcagccccgagtgagtttcgaagtaaatattcagtgctttctttttctttccggaagagtgagaggtggaggtcaaaatggtaggcaagactcgccttcttctttcctttacactgtacagtgatactgaaatatgtacgatttttattataactgttcgtcgcaataaagttatttggagaagtgaggatt

SEQ ID NO: 9 - protein, 522 aa, Zea mays L.
MGACFSSASAAPAGAAVDERRPSKEGDGKKRRRAAGASPDAAAPVRVEFGYERDFEARYEVGRLLGHGQFGYTFAATDRGSGDRVAVKRIDKAKMTRPVAVEDVKREVKILKALKGHQNIVHFYNAFEDDSYVYIVMELCEGGELLDRILAKKNSRYSEKDAAVVVRQMLKVAAECHLRGLVHRDMKPENFLFKSNKEDSPLKATDFGLSDFIKPGKKFHDIVGSAYYVAPEVLKRRSGPESDVWSIGVITYILLCGRRPFWDKTEDGIFKEVLRNKPDFRKRPWSSISPGAKDFVKRLLVKNPRARLTAAQALSHPWVREGGEASDIPVDISVLSNMRQFVKYSRFKQFALRALASTLNEEELSDLKDQFDAIDIDKSGSISIEEMRHALAKDLPWRLKGPRVLEIIQAIDSNTDGLVDFKEFVAATLHIHQMAELDSERWGIRCQAAFSKFDLDGDGYITPEELRMVQHTGLKGSIEPLLEEADIDKDGKISLSEFRKLLRTASMSNVPSPRGPPNPQAL

SEQ ID NO: 10 - DNA, 1940 bp, Zea mays L.
aaatccatatgctcagctcccgcctcctcccatccccggaccccggaccccggccatgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaagatgacccgccctgttgctgtggaggatgtgaaaagagaagtgaagattcttaaagcacttaaaggacatcagaatattgttcacttctacaatgcatttgaggatgattcatacgtgtacattgtgatggagctatgtgagggcggtgaactattagatcggattttggcaaaaaagaatagccgctatagtgagaaagatgctgcagtggtagtccgccaaatgctcaaagtagctgctgaatgccatctgcgtgggttagttcaccgagatatgaagcctgagaacttccttttcaaatcgaacaaggaggattcaccactaaaggcgacagattttggtttgtcagatttcattaagccagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggttctaaggaacaagcctgattttcgtaagaggccttggtcaagcatcagcccaggtgctaaagattttgttaaaaggttactagtgaagaatccaagggccaggctaacagctgctcaagctctctcacatccgtgggtaagagaaggaggggaagcatccgatatccccgtcgacatatctgtgttatcaaacatgcgtcagtttgtcaagtacagccgtttcaagcaattcgcgcttcgggctctggcgagcacccttaacgaggaagagctatcagatctgaaggatcagtttgatgcaattgatatcgataaaagtggatcgattagtatcgaggaaatgcgtcatgcccttgcaaaggatcttccctggagattgaagggtccccgtgtgctggagattattcaagcaattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatggtgcagcacactgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctgtgaattccggctcggccactagggaggagcaagcttaggaagttgccatacaatagccatgtgttctttgggttcttcagagtgccatgtgatgtttctggtttttagcatccaggttatgtgtgcagtgcagccccgagtgagtttcgaagtaaatattcagtgctttctttttctttccggaagagtgagaggtggaggtcaaaatggtaggcaagactcgccttcttctttcctttacactgtacagtgatactgaaatatgtacgatttttattataactgttcgtcgcaataaagttatttggagaagtgagga

SEQ ID NO: 11 - DNA, 287 bp, Zea mays L.
tcaccgagatatgaagcctgagaacttccttttcaaatcgaacaaggaggattcaccactaaaggcgacagattttggtttgtcagatttcattaagccagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggttctaaggaacaagc

SEQ ID NO: 12 - DNA, 13563 bp, Zea mays L.
gggatctgcagctgcttgaatgcagcatggctgatgacgttgagcccagccccaccgtcaatcagaacatggtgcagccgcatgttggtgatgacaagggcgatgatgagcggtaatataccggcccctgccatgttgtcggggcagtcggatgccccaaaagagatagtggtgctcctccaccgttgatgtggagcagccatcgggacccccgggaccgccgaaaggacctctcggcacagggacttgatgttcctgcgggaggtgagctcccagctcccgtcgtatatcatgtacaacttcttgcggcggtcgttgtcatcaccggagtcggagtctccagtgaaaacatccttcaggaccccctcaggagactgatatctgaggtcccgttctcccgcgaccacatcaccgttgtcgaccctctccttgccaggccggcgacgaggtggggagccatccttggaagtctgctcgcaccgctcgctgatgcgcttcgcgagcttgatgatctcgcggcactccgaggcactgtggcgactgttggggtgcacagggcatgagccgctgttgcctccctgtggccgtgggcgcttgttgcgctcgttccggcccccagttgcggctgcgacgatcctagcagtagactgcggcctctcgtggccgcggttcttcttcttcttctttttgccgtcctgggtgacgacacccgagccacccgtcttagcaactccggtttgtggtgtcgagtgccatgcacggccctcggcagctctggcacacttgtcagctagagcgaagagcgtggtgaaagtttccacgtcatgcgtggccaacttctccagcatcttctcatcacgtactctctggcggaaagcggtgatgatggaagcatcgaagatacgaggtatagcgcctcgtaccttggtgaagcgggagatgaaggcccggagagtttccccgggttcctgcctcactgcatggaggtgagcctccatgccatgctgttgataagcactggcgaagttcgctatgaaccacgcgcaaagctcttcccaggagtagatcgatcctagggtgagattcatgagccaggtctgggctggcccagacaaggctacatggaaatatgttgccattacgacagtgtctccacctgctgccgtaatagcggtacgtatacctgcaagaattccgacaggtttgacgtaccgtcgtattttttggcaggtgtggccggaacttggatggccaagtcgccgcgctgagatgatccgctagtgcggcgcagcccacaccggtcaatggaacactcgcctgaattcgggcgcccgttggagtttgcggtgcaaccgcagcgaggtctaggtcgaggttgcgaccctcgatgttctaccggcgctctcgcgccctctccagagagactcgggcatcctcgcccgtacgcctacggttgagttctgctcgcaggtcatctggcctctctagagagactggagcgtcctctcccgcacgcctgcggttgagctccgcccgcaggtcctcagtcggtgcggccttcaccgaggttgagcgcactgacgtcgacgcctcgtgttgacgccgggatgaccgaggcctaggcctcgttgagccagaatgcgccatactgagcagacgatcgacatcatcacaccactgcttcatggcctccggtgaggccgtggagctaggagggtggcgtagcaactccctggctgcagacaaagccccaagtgcagcccttgacgctctggatgtctgcgcaggagtgtgctgccgcgcagagcgcatagcagcagcaccaggcgcatggcggttggaggcccgtggcatggaagacgcagcttcttcctccatatggaagtcctcaggaacgaagtcgtggtgctcgacgatctggaccatggtgtcgagaaggaaaacaggcgaaaacctaatgccaagcccctacctggcgcgccaaatgttggagaggaaaatctccgggcgggtggcggaacgcacccgccctaaatcctaagatgaggagggggcttaagcgtattgcctgtctattagatggtcgatgaacacgagagcacacaaggatttagagtggttcaggccgctggagcgtaataccctactccactgtttgtgtgatgtattgagtctgtgagcttgagagagctcgtgagtctgagttgggtctgccttgtaacgttgtgtgccctcccttttatagctcaaggggggcacatacaaggatgttgagccccgacacgtgggcccagtagcataatgaaagaaatacattataggagtaactaatgcaagtaacgcataagcaatctccggtcgtcgtgatgtccgcagtctatgcagtattgatatgcggcggctgcttccttggtaacgtgcgagtaatgatgagcgtagtgcacgcggcagtatgggcgtgccgcctgccagtggaatggacatttcgccgcctgccagcggaatggataggtctcaataaatgcagaggcggcacaccgtctgccagtggaatggacaggcgacgcgccttatccacaataaatgcagaggtcgcgtagcccagaggccttacgtcaggcttcacccgttggcttacgtcacgtgcagtgtgccacgtggcagcatcggggctctgcctgggcggggagcaggagtgtatgcagacaggtccggacctgcacacgtgtcagcaccggacctcgcttgggtcttgttccaggctcgagtatgttctgtcctagaaccttaggaccccattgtgggcgatccggacccccccacgggggggggggaggggggtacagatcccattctaggggtcctccttgcacacgtggaggtcctggaccaaccttggaggtccggactgtttatcaaggggtccggcgctctcctatgggggtccggacttactgttgatgccttagagtatatcaccttctctagacacgtggcggctccggacccgcccatgtggtggatccgggcgctgctgtggacccagagtagtcgcccgagactggggcaagtcgtggcctcgtcccacacacagcacctttaccacgcgactaagagatagccgcgtgggcactgcgtctttatacaatagtagggggtacccctgtttcagggtactgacaatatcttttttggatttttttatgcgtattttgtccgctcacctaaacatttttaggttatgtttagatatacagtgaaagttaaacatctgaaaaagataacccacaatgttaagtagatggagtactatagtgaaacaaataaatagtcaaagttaatttagaataacaaactcatacaaatcttaaaatgtcattttgtttgtgactatagaaagtattcttagagctaggaggtgcaataaataagacgggcaagtcaacaacctcttcgcttggctttaatttttaccagcttctactatttttataatattttatctctatagaatacaactacatcagtttgaatatctagctttaattcgaggcgaacaatctgaaaaaccttatgcttataagggtagcatcaatggtataggcgtgtatatagcaacgttcatgttagctacatattatactatagcaaagtaatgatatatctatggtagatatttataaatgtttttaatgattaaatacactctctgtaccacctcggtttctgtgtataagtttactccatgttcatagataacagcacactctcacttcattaatttcttgccacatatgatagataccgagagcatctccaatagactagccaaatagaccgtttagccaaattttggctaatcaatagcaaaataactctccaacagactagccatctgactcgtcaaacttttcgactcttcaaattggctccctcactagacaaacctggctagtcactttgactaaccaaactagatagatagtatgctggagtgagttgctatatacagagtgtaatatttatgaaaaaaataaatagagagtcaaataaaaagcaaaaatggagatcccttagagatgttttgacattggttctttatagagagctaattgggtaccgttgcggctgccctaattgaccaagactgaggcaagcaacactagtcactgatgattaggagatgcttgaatgcactaaagctaatagttagttgactaaaaattactaataaaattaactagccaacaaatagctagctaactagttgctaatttattaaaagtagctaataactgaactattagttagactgtttggatgtctcagctaattttaacagctaactattagttctagtgtattaaaacacttccaaataatatgcaagtatttaagataagatcgatgacaacaacatgacgaaataaatcaacctatcaaccaaattgccaggcatcacatgtatcttgtgtggctaaccaaaaggaacgacccggcggtggaggtagagctgaccaagacagcgctgttgccatggagacatttcttttagtggtcttgtttaaacagtaatatttgtatagtaaatgttgttagataaattattgtgaattctattttcgtaatatttacttagatgtgtcatgtgtgtggagtgaccactgagaggtgtttgacaattggcagtttgaccgcttgaccagcggcttcatcatcacatcgtctcgtctgtcgtcgtgtgtggcttcctctcttcccctcctcccttggccccgtctcgtctcctccccctcaccttcctctccaccttcctccctcccacccctccgcgcctcgccccaccgcgccaaccaaccaacacggcgtcccagcctgcctatataccgctccccccgcgcccccacacgcgcaaatccatatgctcagctcccgcctcctcccatccccggaccccggaccccggccatgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaaggtgagctgccgcctgcccccccgcaccccaagccgccgcgctgtccctgtctctgtctctcctactagtagtagtagctggtggtgattccgagcgcgtctttggtctggtgcatcgaaccacttgtgcttggtgcatttcgaggggattcggtgtaattccgtgcaaattggggatttctctcctgttgctttccgaggtttaggtgtttcgattgggacgcgattggagccgttcattttaggacatttccggtgccttttgggaggcgtttagctcaacgagtagctcactcacatttctagctgtttggccgcttcatttctcccaagctttcgttgtttgccggtggttctgagctgcgggatcttgacgttggccagagaggtggtttcgacattcaggcatctcggatgacctcttagtttggcactacagctctattatttcgggaacgacgtgttgctcagtgcgcacctcattcatggaagtggcaaggtcgcttgtctgcagaacggggaaggtgcttttcatctggctattcatggaaaacgacttgttcagttgccctactaataatttcaataagattgcctgcctccttgaatggttggggcttggaaggttcctgtcgaagaaaaagtcaggaaagataacaattgcgcacttgcagtggacaacgcttccctgtcttctatgctataggtggacagcatttttctaggtataattaatttgaccttcaaacatatgtatactaaccaacgcggttttgattccatcaaatgttttggactctctctgctgaactgtcaaagttacttcatggggcaaaatgtcaaattttctggaaccttccgtagtatattttggaaatgagtgtttattgtgtcattggaaataccgttcatgtgtctgtgacagaatgtgtcactagaaagctgaattggtgttgtccttgtcaaaaaggcactaaacacgagtctgaaaattaggcctgttcttggtaagggaaggaatctgagcatcaatgctgataggaatagactctgtctgtcaatattgttaacttgtttatagggcttcgagttttcaacttttgaggcagataagtaggatacctcttttgatcatgatatataacatattcttatatacctcaagccttgcactgttaagttaatgtggcatcctttctagagatcatgacctcaagttgcatatggatgccaataatatcgacaccaagtgaacatcagtgtctgtggaatatgccgaaagcagccaacgtgccattactgaattttcatatgattattatattctgtttagatttatttacgtcggaacacagtgagatggtaacgtaatgaatcaaaataggctataaacatgcaattcaacatatcattatcatgcccaagtgttttgtcattctatctttattcgtccaagaaggacaagcctggtgcattgttgagggaaccagttcttctgcagtacttctagggaggtaaaaattcaacaccgttggatgcagatctatcgaacccagggactttgtgcttccagtgaaaagttatatggacccataggccagaggatgtgagagttttacctctctggaagttatatgcgctagcattagtgtggtcatcaatgggatcaaagatgagctccacctttggtgtagagctggagctaggggactctagcatcctggcgctccaatcttccatccagtgaactctgttttttgggtctagtaggtcaagggtccagttattttttctttctgctgtaaagtctctagttaaggtgtgagttttgtatggtgttttttcgaggtttccccaaacctcaccttttttccttcttaatataatgatatgcagctttcctgcgtattcgagaaaagaaagttttatctctctggaagttaactgcagaggaacttgttacattgttgagagttgtctcaccgagtcaccaggtcgctggttcaaagcagtctctccacatttatgtggaaggcttgcctcggtttatcccttcccaagactctacttgtgggagactctggcattgggtctgtcctatgccgttgaagcgctaggttcgtttatcccttccctatacccacttgtcagagcctccaacactgagtctgccctaagcttccaagttccaacactgggtctgccctaggccgtcgaagcgttatatgattgccatgtactgttatgctttgttgccttcacatattttccgttcgaaatcatctccttgttgccttcacatattgccttgttgctttcacatattttctgttccacgtcatacttagaagttagaacacgtgatttatgccaattaagattattattttatataacagatgacccgccctgttgctgtggaggatgtgaaaagagaagtgaagattcttaaagcacttaaaggacatcagaatattgttcacttctacaatgcatttgaggatgattcatacgtgtacattgtgatggagtaagtaggcccatacacctgttcctgctaatagagcatatcgattttgctatgacttttttccctaaagttttaacatgaacaatatctatcctgtttacagaatcctagacactaaaatgtcatttctaattatcaattattctatagctaaaccagatgcaatcctgatttatttttcttaacgtatggatatattggacttttctttcaaacctgcattttgaatttgattacagggaactataacactaattcagaactctatcatgtttaacatttttcttgcattgttctatgtttgtcaacttgacgcacttcttagataatataacatcatcttccacagtcaccattagttaggaccttggaccttcatggttccgaaatttagctaagaatggtatacatggtcatgtgatttcaaatagatgttcctatatgccagaaccaactcataagtcataagttttaccttgtgtttttgcaggctatgtgagggcggtgaactattagatcggattttggcaaagtaagtagataagatccccatctctttgtttcccgtacctcattcttcgccattaaatttatagatttttgtgctgtaaaatcagattgctttatgttgtttgtctgctttgtttgatttctagttgctcgttcaagatcctttacttaatggtgtgcgtgttttgacagaaagaatagccgctatagtgagaaagatgctgcagtggtagtccgccaaatgctcaaagtagctgctgaatgccatctgcgtgggttagttcaccgagatatgaagcctgaggtagaaatcaaatacttcaatctctttgcacacagtaagcatttggtgatatttcactacttcctcaggtcatgtaagactgtacctattttccttcccagaacttccttttcaaatcgaacaaggaggattcaccactaaaggcgacagattttggtttgtcagatttcattaagccaggtatctacttggggccatctgaatctgtcgggaatctgataggggcaagtctgcagtttagctgaccattttgttgtctaatgcatgctttagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggtaagtggatggattttgcataccatgtgcttacatgtaaaatatgcttggttagagtgctgtaccagggatcagcgttttcagcgtgctgatactgttttgtacaatgtgtttctactttctacgtcatatagcagtgtttctttgttaactatttcagtgtcaaactatttgtcgtgtcacaactcagcagtataattttactattttgaacactgtaaacctgcctggtcaggttatccttcagtaatttctctactagctaccagaaacccactttatgcaggtgttcagtttaataacacccaccatctttcagatttctaatgttcagtgttagacagacttcattaagatgcaccttaagatgattgtaagtagtaaaagtgctttgcacttttgttaacttttgagtctgaagatgacttgtggtacctatgacctcaagaaaccaaggcattgccattggaatagctaattcgaatgagcttcagatatggctatctgttttagttttggacatctgactcaactttataggataatactatattagcaatctttgaggtcattgtctcagccaaaataagttgcggtctcttttttactgtcctaagcagcaatatggtttccattttcattataccagcaacttccacctttttcttgctatttaaatatctttatgcattttatcagcaaggacatgatacgatcgtatatgtgatattctacatcttttcacttctcataattaggttctaaggaacaagcctgattttcgtaagaggccttggtcaagcatcagcccaggtgctaaagattttgttaaaaggttactagtgaagaatccaagggccaggctaacagctgctcaagctctctgtaagttttggtatttttcattaatttactagcctagtcatgatgatcagattcaccttctctatgtgagaacagagaacacatatacatctggcagtatgcctttcaatcagttatgacaatgtaaatatgcaaagaccgatgttttttctatcctgcaccattttagaacattaatggggaaaaaccacaatatattaggaaaaatgtttaattatgtcctggtcacttgaaatgaacatataccactgaggttttctagttctcatgcgttcttataatgatctaataagtcagtggaggtttgctgcccaccacccctacatttgtattgtgaattactatcatctttactgatcctgattgttcttgatatgttaagcacatccgtgggtaagagaaggaggggaagcatccgatatccccgtcgacatatctgtgttatcaaacatgcgtcagtttgtcaagtacagccgtttcaagcaattcgcgcttcgggtaattacagtgattacaaaaaacaacactgcatcgtttattttttcctcacaatatttcctcgtggcatggtcaggctctggcgagcacccttaacgaggaagagctatcagatctgaaggatcagtttgatgcaattgatatcgataaaagtggatcgattagtatcgaggaaatgcgtcatgtaggttctgttagtgtttgctgatgaaaatgccttagatcctgaactactctgcggtgctgattaatctgtgcatgtttcggtaggcccttgcaaaggatcttccctggagattgaagggtccccgtgtgctggagattattcaagcagtaagtttgagccttcttctggatccagccctttctttgttaccccccttgtttccaagaaaatagctggccttgttctgagggtataaccaaaactgcatcttattttgtggtagattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatggtaattttctactcctgtcttgtttccatgttgcttcaccaacgaatgcacagttcacataacccttattatcatcactgcttcccatgaataactagctggctcgaccatcatgagattcagtacttgcgccctgtgcacttggttttggtcccgcttgttagaatgaagtaatttatcaatggaagcgctgtaatattttaatcagcgtttagatttgataaagataaaacatgttcattgtttgtgccaagaaatccacttacacagatactgagagttgcaccgtagataacgctaatcggcagtatcctaatcgagattttctttcaaggtgcagcaccctgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctgtgaattccggctcggccactagggaggagcaagcttaggaagttgccatacaatagccatgtgttctttgggttcttcagagtgccatgtgatgtttctggtttttagcatccaggttatgtgtgcagtgcagccccgagtgagtttcgaagtaaatattcagtgctttctttttctttccggaagagtgagaggtggaggtcaaaatggtaggcaagactcgccttcttctttcctttacactgtacagtgatactgaaatatgtacgatttttattataactgttcgtcgcaataaagttatttggagaagtgaggattttattgtcctggtgaacctgtacgttttttccccaaacggatcacggtcacggcccccaagtttcagcataaaaagtttatgacagatttctggtttggttcgctgcctcattcatgctgttcgtttgggagtaaaactagttgttgcaactgtaatctatagagacaattagataaaatactgagattaaagctaaatgaacagactgccacaatttattatgtttaaggttagatatttaatcgatttaggcgtgtgtttggtttatagctataattgtgatattttttttttctgttgtgtagattttacttgtcaacaattgtgatttttttcttctgttgtgtagattttacttgtcaacaatttattatgtttaaggttagacatttaatcgatttagacgtgtgtttggtttatagctataattgtgatattttttttctgttgtgtagatttcattgtaaaagtgtgatagttttttaggtttttctaaatgtagtgaagaatttgagcgcagacaggtgtggcagaaaactgtgtaccaccaaacagccccttcatttgcagttccattgccgcgacaagcaattgcggttgatgattgaggatccggtggcagtacagcagactgcccgttcggaaaagcgcgcgggggcttctccctagtcgcttggcaggctgaatgccggcaccgcggttggcgttaaacccggccgccgtgctccacgcggcgctcctcagggcctcctctgccggttgccgcctgcctccccgcatctccttcaactcgctgctggcggccgccgcgtcctccgcggacacgcgcacccgcgctctcgcgctcccggctctcgcgctcgcccacgcctccggccgcgtgtccctcgactcgtacgccctctgctccgcgctccgctccgcgccctccgccgcggggacgctgcacgcgctggccgccaagtccggctggctcggcagcgtcttcgtgtcctgcgcgctcgccgcttcctacggcgggtccggccggtgcctggacgcccggagcctgttcgacgaaagtcccgccaggaacggcgtcttcgggaacgccgtcctcgccgcttacgtgggcgcggccgagtgggctcccgtgctgaggttcgccaggaggttctcggaactgcggctgcaggttgactggtacacgatgacggctgtggcgcgggcgtgtggcgaggtggccaacgctgatctcggcgtccaggcgcatgggcatgcggtcaggaggctgggaggtgtagaggtggacgtgttcttggtcagcgcgttcgtggacatgtacgccaagtgcgggcttatcagccaagcggagcgtgtgttccgccttgcgcaacaggagaccggtggcagaggtgacgttgtgctgtggacggccatgttgaacgcctatgcgcggcatggacagtgcaaggaggttatccggcagtatgacctgatgctggcctctggtgtctatccggatgaattggccatgttagctgtactctcagcttgccagcacgccggggaggtggtcaaggggctcaactactttgaatccatgcatgcagattacgggctggtgcccacaccggagcactacggttgtgtggtcaacatgctgtgccgggcaggggaagtgaccaaggcgtgggagattgccaccaaggacggctgtgatc

SEQ ID NO: 13 - DNA, 2331 bp, Artificial sequence
  modified gene construct
atgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaagatgacccgccctgttgctgtggaggatgtgaaaagagaagtgaagattcttaaagcacttaaaggacatcagaatattgttcacttctacaatgcatttgaggatgattcatacgtgtacattgtgatggagctatgtgagggcggtgaactattagatcggattttggcaaaaaagaatagccgctatagtgagaaagatgctgcagtggtagtccgccaaatgctcaaagtagctgctgaatgccatctgcgtgggttagttcaccgagatatgaagcctgagaacttccttttcaaatcgaacaaggaggattcaccactaaaggcgacagattttggtttgtcagatttcattaagccagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggttctaaggaacaagcctgattttcgtaagaggccttggtcaagcatcagcccaggtgctaaagattttgttaaaaggttactagtgaagaatccaagggccaggctaacagctgctcaagctctctcacatccgtgggtaagagaaggaggggaagcatccgatatccccgtcgacatatctgtgttatcaaacatgcgtcagtttgtcaagtacagccgtttcaagcaattcgcgcttcgggctctggcgagcacccttaacgaggaagagctatcagatctgaaggatcagtttgatgcaattgatatcgataaaagtggatcgattagtatcgaggaaatgcgtcatgcccttgcaaaggatcttccctggagattgaagggtccccgtgtgctggagattattcaagcaattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatgcaccctgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctggatccggctgctgccgctgccgctgcggcagcggccggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 14 - DNA, 2337 bp, Artificial sequence
  modified gene construct
atgggcgcttgcttctcctccgcctctgccgcccccgccggcgccgccgtcgacgagcgccgcccgtccaaggagggcgacggcaagaagaggcgccgcgccgccggggcatcgccggatgccgcggcgcccgtgcgcgtggagttcggctacgagagggacttcgaggcgcgctacgaggtcggccgcctgctcggccacggccagttcggctacaccttcgccgccaccgaccgcggctctggggaccgcgttgccgtcaagcgcatcgacaaggccaagatgacccgccctgttgctgtggaggatgtgaaaagagaagtgaagattcttaaagcacttaaaggacatcagaatattgttcacttctacaatgcatttgaggatgattcatacgtgtacattgtgatggagctatgtgagggcggtgaactattagatcggattttggcaaaaaagaatagccgctatagtgagaaagatgctgcagtggtagtccgccaaatgctcaaagtagctgctgaatgccatctgcgtgggttagttcaccgagatatgaagcctgagaacttccttttcaaatcgaacaaggaggattcaccactaaaggcgacagattttggtttgtcagatttcattaagccagggaagaagttccatgacattgttggaagtgcttactatgtcgcaccagaagtactaaaacgacggtctggtcctgagtcagatgtttggagcataggagtcataacctacattttgctctgtgggaggcgccctttttgggataagaccgaagacggtatattcaaggaggttctaaggaacaagcctgattttcgtaagaggccttggtcaagcatcagcccaggtgctaaagattttgttaaaaggttactagtgaagaatccaagggccaggctaacagctgctcaagctctctcacatccgtgggtaagagaaggaggggaagcatccgatatccccgtcgacatatctgtgttatcaaacatgcgtcagtttgtcaagtacagccgtttcaagcaattcgcgcttcgggctctggcgagcacccttaacgaggaagagctatcagatctgaaggatcagtttgatgcaattgatatcgataaaagtggatcgattagtatcgaggaaatgcgtcatgcccttgcaaaggatcttccctggagattgaagggtccccgtgtgctggagattattcaagcaattgacagcaacactgatgggctcgtggacttcaaggagtttgttgcggcaactctccatatccaccagatggcggagctcgactcagaaaggtggggcatacgctgccaagctgctttcagtaagtttgatcttgacggtgatggatatatcacgccggaggaactcagaatggtgcagcaccctgggttgaagggatctatcgagccgctgctggaggaggccgacatcgacaaagacggcaagataagcctgtccgagttccgcaagctcctacggacagcgagcatgagcaacgtacccagcccaagggggcccccaaaccctcaggctctggatccggctgctgccgctgccgctgcggcagcggccggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 15 - protein, 239 aa, Artificial sequence
  EGFP peptide tag
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 16 - DNA, 953 bp, Artificial sequence
  modified gene construct
ggtctctggcgacaagcctgattttcgtaaggttttagagctagaaatagcaagttaaaataaggctagtccgttatcaacttgaaaaagtggcaccgagtcggtgcttttttttttcgttttgcattgagttttctccgtcgcatgtttgcagttttattttccgttttgcattgaaatttctccgtctcatgtttgcagcgtgttcaaaaagtacgcagctgtatttcacttatttacggcgccacattttcatgccgtttgtgccaactatcccgagctagtgaatacagcttggcttcacacaacactggtgacccgctgacctgctcgtacctcgtaccgtcgtacggcacagcatttggaattaaagggtgtgatcgatactgcttgctgctcatgaatccaaaccacacggagttcaaattcccacagattaaggctcgtccgtcgcacaaggtaatgtgtgaatattatatctgtcgtgcaaaattgcctggcctgcacaattgctgttatagttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagcccaccaacctcacgggccagcaattacgagtccttctaaaagctcccgccgaggggcgctggcgctgctgtgcagcagcacgtctaacattagtcccacctcgccagtttacagggagcagaaccagcttataagcggaggcgcggcaccaagaagctgatgggctcgtggacttcagtttagagacc

SEQ ID NO: 17 - RNA, 20 nt, Artificial sequence
  primer
acaagccugauuuucguaag

SEQ ID NO: 18 - RNA, 20 nt, Artificial sequence
  primer
ugaugggcucguggacuuca

SEQ ID NO: 19 - protein, 17 aa, Artificial sequence
  peptide fragment
DPAAAAAAAAAAGPVAT

SEQ ID NO: 20 - protein, 4 aa, Artificial Sequence
  peptide tag
RRRR

SEQ ID NO: 21 - protein, 6 aa, Artificial Sequence
  peptide tag
HHHHHH

SEQ ID NO: 22 - protein, 8 aa, Artificial Sequence
  peptide tag
DYKDDDDK

SEQ ID NO: 23 - protein, 8 aa, Artificial Sequence
  peptide tag
WSHPQFEK

SEQ ID NO: 24 - protein, 10 aa, Artificial Sequence
  peptide tag
EQKLISEEDL

SEQ ID NO: 25 - protein, 9 aa, Artificial Sequence
  peptide tag
YPYDVPDYA

SEQ ID NO: 26 - DNA, 20 bp, Artificial Sequence
  ZmPK primer
atgggcgcttgcttctcctc

SEQ ID NO: 27 - DNA, 21 bp, Artificial Sequence
  ZmPK primer
tcacagagcctgagggtttgg

SEQ ID NO: 28 - DNA, 21 bp, Artificial Sequence
  Bar primer
gaaggcacgcaacgcctacga

SEQ ID NO: 29 - DNA, 21 bp, Artificial Sequence
  Bar primer
ccagaaacccacgtcatgcca

SEQ ID NO: 30 - DNA, 19 bp, Artificial Sequence
  ZmPK primer
gcgttgccgtcaagcgcat

SEQ ID NO: 31 - DNA, 21 bp, Artificial Sequence
  ZmPK primer
gctccatcacaatgtacacgt

SEQ ID NO: 32 - DNA, 20 bp, Artificial Sequence
  GAPDH primer
atcaacggcttcggaaggat

SEQ ID NO: 33 - DNA, 20 bp, Artificial Sequence
  GAPDH primer
ccgtggacggtgtcgtactt

SEQ ID NO: 34 - DNA, 20 bp, Artificial Sequence
  primer
ttgaggtcattgtctcagcc

SEQ ID NO: 35 - DNA, 22 bp, Artificial Sequence
  primer
agcagcttggcagcagcgtatg

(19) United States
(12) Patent Application Publication
(10) Pub. No.: US 2022/0170041 A1
(43) Pub. Date: Jun. 2, 2022

(54) METHOD FOR CULTIVATING PLANT RESISTANT TO GRAY LEAF SPOT

(71) Applicant: CHINA AGRICULTURAL UNIVERSITY, Beijing (CN)

(72) Inventors: Mingliang XU, Beijing (CN); Mang ZHU, Beijing (CN); Xingming FAN, Beijing (CN); Tao ZHONG, Beijing (CN); Ling XU, Beijing (CN); Yan ZHANG, Beijing (CN); Li LIU, Beijing (CN)

(21) Appl. No.: 17/434,206

(22) PCT Filed: Feb. 24, 2020
(86) PCT No.: PCT/CN2020/076319

§ 371 Date: Aug. 26, 2021

(30) Foreign Application Priority Data
Mar. 4, 2019 (CN) 201910160206.3

(51) Int. Cl. C12N 15/82; C07K 14/415

(52) U.S. Cl. CPC C12N 15/8282; C07K 14/415

(57) ABSTRACT

The present invention discloses a method for cultivating a plant resistant to gray leaf spot. The proteins provided by the present invention are obtained from corn and named as ZMPK protein, and are the proteins represented by seq. 2, seq. 4, seq. 7 or seq. 9 in the sequence list. Nucleic acid molecules encoding the ZMPK proteins are also within the scope of the present invention. The invention further sets forth a method for preparing a transgenic plant, comprising the step of: introducing the nucleic acid molecules into a starting plant to obtain the transgenic plant with reduced resistance to gray leaf spot. The invention further sets forth a method for preparing a transgenic plant, comprising the step of: knocking out or inhibiting the expression of the nucleic acid molecules in a starting plant to obtain the transgenic plant with increased resistance to gray leaf spot. The present invention is of great application value to the breeding of corn resistant to gray leaf spot.

### TECHNICAL FIELD

The present invention belongs to the field of biotechnology, and specifically relates to a method for cultivating plants resistant to gray leaf spot; more specifically, it relates to a method for genetically improving plants by expressing a ZmPK gene to obtain plants resistant to gray leaf spot.

### BACKGROUND ART

Corn gray leaf spot is a worldwide fungal disease of corn leaves. It was first discovered in Alexandria County, Ill., USA in 1925, and seriously affected the yield of corn. In China, the corn gray leaf spot first occurred in Dandong City, Liaoning Province in 1991. After that, it was reported in Jilin, Hebei, Yunnan and other regions. In recent years, gray leaf spot has become one of the main leaf diseases in corn production in China, especially in the southwest corn producing areas. The occurrence of corn gray leaf spot generally causes a 10-30% reduction in corn production, and in severe cases, it can reach 60-80% or even no harvest, which severely affects the production of corn in China.

Current researchers believe that there are two main pathogens causing corn gray leaf spot: Cercospora zeae-maydis (Czm) and Cercospora zeina (Cz). For a long time, domestic researchers believed that the pathogen of corn gray leaf spot in China was Czm. Liu et al. took samples in the Yunnan area and analyzed the morphology, pathogenicity, ITS sequence and histone H3 gene sequence of the microorganism and found that the pathogen of corn gray leaf spot in the Yunnan area in China is Cz. The disease spots first appeared on the lower leaves, and the symptoms were most obvious on the leaves. In the early stage of onset, the disease spots were water-stained faded-green spots, and then expanded to grayish-brown color spots, which were approximately rectangular and parallel to the veins of the leaves. When the disease is severe, the disease spots expand and spread, causing the leaves to wither. The use of fungicides and other chemical control methods is not effective. Breeding varieties resistant to gray leaf spot is the most economical and effective way to control this disease.

### SUMMARY OF THE INVENTION

The present invention provides a method for cultivating plants resistant to gray leaf spot.

The present invention provides a protein, which is obtained from corn, and named as ZmPK protein, which is as follows: (a1), or (a2), or (a3), or (a4), or (a5), or (a6), or (a7), or (a8):

(a1) a protein represented by SEQ ID NO: 2 in the sequence listing;

(a2) a protein represented by SEQ ID NO: 4 in the sequence listing;

(a3) a protein represented by SEQ ID NO: 7 in the sequence listing;

(a4) a protein represented by SEQ ID NO: 9 in the sequence listing;

(a5) a fusion protein obtained by attaching a tag to an N-terminus or/and a C-terminus of the protein in any one of (a1) to (a4);

(a6) a protein comprising the following three segments from N-terminus to C-terminus: the protein in any one of (a1) to (a4), a connecting peptide, and an EGFP protein;

(a7) a protein related to plant gray leaf spot resistance obtained by substituting and/or deleting and/or adding one or a plurality of amino acid residues to the protein in any one of (al) to (a6); and

(a8) a protein related to plant gray leaf spot resistance obtained from corn and having a homology of 90%, 91%, 92%, 93%, 94%, 95%, 96%, 97%, 98% or 99% with the protein in any one of (a1) to (a4).

The tags are as shown in Table 1 below.

TABLE 1
Tag sequences

| Tag | Residues | Sequence |
|---|---|---|
| Poly-Arg | 5 to 6 (typically 5) | RRRR |
| Poly-His | 2 to 10 (typically 6) | HHHHHH |
| FLAG | 8 | DYKDDDDK |
| Strep-tag II | 8 | WSHPQFEK |
| c-myc | 10 | EQKLISEEDL |
| HA | 9 | YPYDVPDYA |
| EGFP | 239 | Sequence 15 |

The EGFP protein is specifically shown in Sequence 15 in the sequence Listing. The connecting peptide can be specifically as shown in Sequence 19 in the sequence listing.

The protein can be synthesized artificially, or its encoding gene can be synthesized first, and then the protein can be obtained by biological expression.

The nucleic acid molecule encoding the ZmPK protein also falls within the scope of protection of the present invention.

The nucleic acid molecule is any one of the following (b1) to (b15):

(b1) a DNA molecule with an encoding region that is represented by nucleotides 56 to 1618 in SEQ ID NO: 3 in the sequence listing;

(b2) a DNA molecule represented by SEQ ID NO: 3 in the sequence listing;

(b3) a DNA molecule with an encoding region that is represented by nucleotides 56 to 1624 in SEQ ID NO: 5 in the sequence listing;

(b4) a DNA molecule represented by SEQ ID NO: 5 in the sequence listing;

(b5) a DNA molecule represented by SEQ ID NO: 1 in the sequence listing;

(b6) a DNA molecule with an encoding region that is represented by nucleotides 56 to 1618 in SEQ ID NO: 8 in the sequence listing;

(b7) a DNA molecule represented by SEQ ID NO: 8 in the sequence listing;

(b8) a DNA molecule with an encoding region that is represented by nucleotides 56 to 1624 in SEQ ID NO: 10 in the sequence listing;

(b9) a DNA molecule represented by SEQ ID NO: 10 in the sequence listing;

(b10) a DNA molecule represented by SEQ ID NO: 6 in the sequence listing;

(b11) a DNA molecule represented by SEQ ID NO: 12 in the sequence listing;

(b12) a DNA molecule represented by SEQ ID NO: 13 in the sequence listing;

(b13) a DNA molecule represented by SEQ ID NO: 14 in the sequence listing;

(b14) a DNA molecule that is derived from corn, has a homology of 90%, 91%, 92%, 93%, 94%, 95%, 96%, 97%, 98% or 99% with any one of (b1) to (b13), and encodes the protein;

(b15) a DNA molecule that hybridizes to any one of (b1) to (b13) under a stringent condition, and encodes the protein.

The stringent condition mentioned above is as follows: in a solution of 2× SSC, 0.1% SDS, hybridizing is performed at 68° C., and the membrane is washed twice for 5 min each time, and then hybridizing is performed again in a solution of 0.5× SSC, 0.1% SDS at 68° C., and the membrane is then washed twice for 15 min each time.

An expression cassette, recombinant vector or recombinant microorganism containing the nucleic acid molecule also falls within the scope of protection of the present invention.

The existing expression vectors can be used to construct a recombinant expression vector containing the nucleic acid molecule. When using the nucleic acid molecule to construct a recombinant expression vector, any enhanced, constitutive, tissue-specific or inducible promoter can be added before its transcription initiation nucleotide, and they can be used alone or in combination with other plant promoters that can be used in combination. In addition, when using the nucleic acid molecule to construct a recombinant expression vector, enhancers, including translation enhancers or transcription enhancers, can also be used. These enhancer regions can be ATG start codons or adjacent region start codons, etc., but they must be in the same reading frame with the coding sequence in order to ensure correct translation of the entire sequence. The sources of the translation control signals and initiation codons are extensive, and they can be natural or synthetic. The translation initiation region can be derived from a transcription initiation region or a structural gene. In order to facilitate the identification and screening of transgenic plants or transgenic microorganisms, the expression vectors used herein can be processed. For example, gene expressing enzymes or luminescent compounds that can produce color changes in plants or microorganisms, resistant antibiotic markers or chemical reagent resistant marker genes, etc. can be added herein. Considering the safety of the transgenes, it is possible to directly screen transformed plants or microorganisms by phenotype without adding any selectable marker genes.

The recombinant expression vector may specifically be a recombinant plasmid obtained by inserting the double-stranded DNA molecule shown in Sequence 12 in the sequence listing into the multiple cloning site (for example, the BamHI site) of the pCAMBIA3301 vector.

The recombinant expression vector may specifically be a recombinant plasmid obtained by inserting the nucleic acid molecule into the multiple cloning site (for example, the XcmI restriction site) of the pBCXUN vector.

The present invention further sets forth the application of the ZmPK protein, which is the following (c1) or (c2): (c1) to regulate the resistance of a plant to gray leaf spot; and (c2) to reduce the disease resistance of a plant to gray leaf spot.

The present invention further sets forth the application of the nucleic acid molecule, which is the following (d1) or (d2): (d1) to cultivate a transgenic plant with altered resistance to gray leaf spot; and (d2) to cultivate a transgenic plant with reduced resistance to gray leaf spot.

The application of the nucleic acid molecule further includes using the nucleic acid molecule as a target to reduce the expression amount of the nucleic acid molecule. The implementation methods include, but are not limited to: RNAi interference, gene knockout, etc.

The implementation methods also include: insertion, deletion or editing of the promoter region, and promoter interchange. The methods for the target may also include, but are not limited to: using editing or mutant alleles with lower expression or weaker activity, etc.

The present invention further sets forth an application of a substance for inhibiting an activity of the ZmPK protein in a plant and/or for reducing an abundance of the ZmPK protein in a plant to enhance the disease resistance of the plant to gray leaf spot.

The present invention further sets forth an application of a substance for inhibiting a transcription of the nucleic acid molecule and/or for inhibiting an expression of the nucleic acid molecule and/or for gene editing of the nucleic acid molecule to enhance the disease resistance of the plant to gray leaf spot. The “substance for gene editing of the nucleic acid molecule” may specifically be any interference vector described later or any gene editing vector described later.

The present invention further sets forth a method for preparing a transgenic plant, comprising the steps of: introducing the nucleic acid molecule into a starting plant to obtain a transgenic plant with reduced gray leaf spot resistance. The nucleic acid molecule can be specifically introduced into the starting plant through any one of the above-mentioned recombinant expression vectors. The recombinant expression vector carrying the nucleic acid molecule can be transformed into the starting plant by conventional biological methods such as Ti plasmid, Ri plasmid, plant virus vector, direct DNA transformation, microinjection, electrical conduction, and Agrobacterium mediation. By crossing the transgenic plants with existing corn varieties (including single crosses and multiple crosses, such as three consecutive crosses), the obtained transgenic progeny plants are also transgenic plants with reduced gray leaf spot resistance. The existing corn variety may specifically be a corn inbred line Q11.

The present invention further sets forth a plant breeding method, comprising the following steps: increasing a content and/or activity of the ZmPK protein in a target plant, thereby reducing the gray leaf spot resistance of the target plant.

The present invention further sets forth a plant breeding method, comprising the following steps: inhibiting an expression of the nucleic acid molecule in a starting plant to obtain a transgenic plant with increased gray leaf spot resistance. Inhibiting the expression of the nucleic acid molecule in the starting plant can be specifically achieved by means of introducing an interference vector. The interference vector may specifically be the following recombinant plasmid: a recombinant plasmid having a forward fragment, a spacer fragment and a reverse fragment; the spacer segment is used to space the forward segment and the reverse segment; the forward segment and the reverse segment are in a reverse complementary relationship; the forward fragment is shown in Sequence 11 in the sequence listing. The interference vector may specifically be the following recombinant plasmid: a recombinant plasmid obtained by using the pGreen-HY104 vector as a starting vector, and inserting forward fragments and reverse fragments into different multiple cloning sites; the forward segment and the reverse segment are in a reverse complementary relationship; and the forward fragment is shown in Sequence 11 in the sequence listing. The interference vector may specifically be the following recombinant plasmid: an RNAi interference vector obtained by using the pGreen-HY104 vector as the starting vector, inserting a forward fragment between the BamHI and XbaI restriction sites, and inserting a reverse fragment between the HindIII and EcoRI restriction sites; the forward fragment and the reverse fragment are in a reverse complementary relationship; and the forward fragment is shown in Sequence 11 in the sequence listing. The interference vector can be transformed into the starting plant by conventional biological methods such as Ti plasmid, Ri plasmid, plant virus vector, direct DNA transformation, microinjection, electric conduction, Agrobacterium mediation and the like. By crossing the transgenic plants with existing corn varieties (including single crosses and multiple crosses, such as three consecutive crosses), the obtained transgenic progeny plants are also transgenic plants with reduced gray leaf spot resistance. The existing corn variety may specifically be a corn inbred line Q11.

The present invention further sets forth a plant breeding method, comprising the following steps: reducing a content and/or activity of the protein ZmPK in a target plant, thereby increasing the disease resistance of the target plant to gray leaf spot.

The present invention further sets forth a plant breeding method, comprising the steps of: performing gene editing (causing a frameshift mutation in the specific gene) on a specific gene in a genome of a starting plant to increase the gray leaf spot resistance of the target plant; and the specific gene encoding the ZmPK protein.

The gene editing is specifically realized by the Cas9 technology.

The gene editing is specifically realized by two sgRNAs and the Cas9 protein, in which the target sequence binding region of one sgRNA is shown in Sequence 17 of the sequence listing, and the target sequence binding region of the other sgRNA is shown in Sequence 18 of the sequence listing.

The gene editing is specifically realized by introducing a gene editing vector. The gene editing vector may specifically be the following recombinant plasmid: a recombinant plasmid obtained by inserting the double-stranded DNA molecule shown in Sequence 16 in the sequence listing into the BsaI restriction site of the pBUE411 vector.

Any of the above-mentioned plants is a dicotyledonous plant or a monocotyledonous plant. The monocotyledonous plant may be a gramineous plant. The gramineous plant may be a plant of the genus Zea. The Zea plant may specifically be corn, such as corn inbred line B73 or corn inbred line B73-329.

Any of the above gray leaf spots may specifically be gray leaf spot caused by Cercospora comae.

### BRIEF DESCRIPTION OF THE DRAWINGS

FIG. 1 is a schematic diagram of the structure of a pBCXUN vector.

FIG. 2 is the results of the identification of gene expression level in Example 2.

FIG. 3 is the results of the identification of disease resistance in Example 2.

FIG. 4 is a schematic diagram of the structure of an RNAi interference vector.

FIG. 5 is the results of the identification of gene expression level in Example 3.

FIG. 6 is the results of the identification of disease resistance in Example 3.

FIG. 7 is the results of the correlation between the expression level of the ZmPK gene and the level of gray leaf spot.

FIG. 8 is a schematic diagram of the structure of a pCAMBIA3301 vector.

FIG. 9 is the results of the identification of gene expression level in Example 5.

FIG. 10 is the results of the identification of disease resistance in Example 5.

FIG. 11 is a schematic diagram of the structure of recombinant plasmid E and recombinant plasmid F.

FIG. 12 is the results of the identification of gene expression level in Example 6.

FIG. 13 is the results of the identification of disease resistance in Example 6.

FIG. 14 is the sequencing results of two gene-edited plants.

FIG. 15 is the results of the identification of disease resistance in Example 7.

### DESCRIPTION OF THE PREFERRED EMBODIMENTS

The following examples facilitate a better understanding of the present invention, but do not limit the present invention. The experimental methods in the following examples, unless otherwise specified, are all conventional methods. The experimental materials used in the following examples, unless otherwise specified, are all purchased from conventional biochemical reagent stores. The quantitative experiments in the following examples are all set to repeat the experiment three times, and the results are averaged.

The corn inbred line Y32 is a corn inbred line with high resistance to gray leaf spot of corn. The corn inbred line Y32 (line Y32) is described in the following documents: QTL mapping of resistance to gray leaf spot in maize, Yan Zhang etc., Theor Appl Genet DOI 10.1007/s00122-012-1954-z.

The corn inbred line Q11 is a corn inbred line highly susceptible to gray leaf spot. The corn inbred line Q11 (line Q11) is described in the following documents: QTL mapping of resistance to gray leaf spot in maize, Yan Zhang ect., Theor Appl Genet DOI 10.1007/s00122-012-1954-z.

The corn inbred line B73 (B73 inbred lines) is described in the following documents: The B73 maize genome: complexity, diversity, and dynamics, Patrick S. Schnable ect., Science (2009) 326: 1112-1115. DOI: 10.1126/science. 1178534; The tin1 gene retains the function of promoting tillering in maize, Zhang ect., Nature communications (2019) 5608. DOI: 10.1038/s41467-019-134225-6.

The corn inbred line B73-329 (B73-329 inbred lines) is described in the following documents: A retrotransposon in an HKT1 family sodium transporter causes variation of leaf Na⁺ exclusion and salt tolerance in maize, Ming Zhang ect., New Phytologist (2018) 217: 1161-1176 doi: 10.1111/nph.14882.

Cercospora zeina is described in the following documents: First Report of Gray Leaf Spot of Maize Caused by Cercospora zeina in China, Plant Disease/Vol. 97 No. 12.

The pBCXUN vector is described in the following documents: ZmHAK5 and ZmHAK1 function in K⁺ uptake and distribution in maize under low K+conditions. Journal of Intergrative

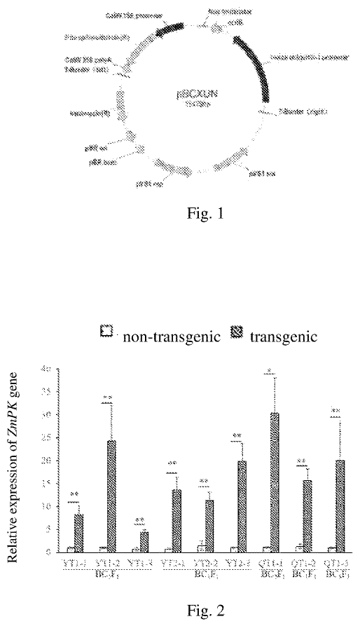

Plant Biology (2018) doi: 10.1111/jipb. 12756. The schematic diagram of the structure of a pBCXUN vector is shown in FIG. 1.

The pGreen-HY104 vector (vector pGreen-HY104) is described in the following documents: A maize wall-associated kinase confers quantitative resistance to head smut, Nature Genetics (2015) 47: 151-157.

The pCAMBIA3301 vector (bivalent expression vector pCAMBIA3301) is described in the following documents: Pyramiding of nine transgenes in maize generates high-level resistance against necrotrophic maize pathogens.

The pBUE411 vector is described in the following documents: ZmCCT9 enhances maize adaptation to higher latitudes. Huang ect., PNAS (2018) 115(2): E334-E341 DOI: 10.1073/pnas.1718058115.

### EXAMPLE 1

### Discovery of ZmPK Protein and its Encoding Gene

The corn inbred line Y32 (as the donor parent) and the corn inbred line Q11 (as the recurrent parent) were used to construct the initial positioning population and the fine positioning population. The molecular markers located in the finely-located regions were used; the Y32BAC library of disease-resistant parents was screened by PCR. BAC clone fingerprint analysis was performed to construct BAC contigs covering the entire gene segment. The clone that could cover the least gene region was then selected for sequencing. A new gene was discovered through sequence alignment and expression analysis.

The ZmPK gene in the genomic DNA of the corn inbred line Y32 is shown in Sequence 1 in the sequence listing. There are two transcripts of the ZmPK gene. The first transcript encodes the protein shown in Sequence 2 in the sequence listing; the second transcript encodes the protein shown in Sequence 4 in the sequence listing. The open reading frame corresponding to the first transcript in the cDNA of the corn inbred line Y32 is shown in nucleotides 56-1618 in Sequence 3 in the sequence listing. The open reading frame corresponding to the second transcript in the cDNA of the corn inbred line Y32 is shown in nucleotides 56-1624 in Sequence 5 in the sequence listing.

The ZmPK gene in the genomic DNA of the three corn inbred lines (the three corn inbred lines refer to the corn inbred line B73, the corn inbred line B73-329 and the corn inbred line Q11, the same below) is as shown in Sequence 6 in the sequence listing. There are two transcripts of the ZmPK gene. The first transcript encodes the protein shown in Sequence 7 in the sequence listing. The second transcript encodes the protein shown in Sequence 9 in the sequence listing. The open reading frame corresponding to the first transcript of the cDNAs of the three corn inbred lines is shown in nucleotides 56-1618 in Sequence 8 in the sequence listing. The open reading frame corresponding to the second transcript of the cDNAs of the three corn inbred lines is shown in nucleotides 56-1624 in Sequence 10 in the sequence listing.

### EXAMPLE 2

### Obtaining and Identifying Over-Expression Plants

### I. Construction of Recombinant Expression Vector

1. Fresh leaves of corn inbred line Y32 were taken, total RNA was extracted, and cDNA was obtained by reverse transcription.

2. The cDNA obtained in step 1 was used as a template; a primer pair composed of ZmPK-OE-F and ZmPK-OE-R was used for PCR amplification to obtain a PCR amplification product.

ZmPK-OE-F:
5′-ATGGGCGCTTGCTTCTCCTC-3′;
ZmPK-OE-R:
5′-TCACAGAGCCTGAGGGTTTGG-3′.

3. The pBCXUN vector was taken, cleaved with restriction enzyme XcmI, and the vector backbone was recovered.

4. The PCR amplification product obtained in step 2 was linked to the vector backbone obtained in step 3 to obtain a recombinant plasmid A and a recombinant plasmid B. According to the sequencing results, the structure of recombinant plasmid A was described as follows: at the XcmI restriction site of the pBCXUN vector, a DNA molecule represented by nucleotides 56-1618 in Sequence 3 in the sequence listing was inserted. According to the sequencing results, the structure of recombinant plasmid B was described as follows: at the XcmI restriction site of the pBCXUN vector, a DNA molecule represented by nucleotides 56-1624 in Sequence 5 in the sequence listing was inserted. Since there were two sequences in the template and the difference was only 6 nucleotides, and the recombinant plasmid A and the recombinant plasmid B were constructed in the same way and produced at the same time, they needed to be identified by sequencing. In practical applications, two exogenous fragments can also be directly synthesized and then inserted into the XcmI restriction site of the pBCXUN vector to obtain a recombinant plasmid A and a recombinant plasmid B.

5. Fresh leaves of corn inbred line Q11 were taken, total RNA was extracted, and a cDNA was obtained by reverse transcription.

6. The cDNA obtained in step 5 was used as a template; a primer pair composed of ZmPK-OE-F and ZmPK-OE-R was used for PCR amplification to obtain a PCR amplification product.

7. The PCR amplification product obtained in step 6 was linked to the vector backbone obtained in step 3 to obtain a recombinant plasmid C. According to the sequencing results, the structure of recombinant plasmid C was described as follows: at the XcmI restriction site of the pBCXUN vector, a DNA molecule represented by nucleotides 56-1618 in Sequence 8 in the sequence listing was inserted.

### II. Acquisition of an Overexpression Plant

1. The recombinant plasmid was introduced into Agrobacterium EHA105 to obtain recombinant Agrobacterium.

2. The recombinant Agrobacterium obtained in step 1 was taken, and the Agrobacterium-mediated method was used to genetically transform the immature embryos of the corn inbred line B73-329 to obtain the T0 generation plants.

3. The T0 generation plants were identified by PCR.

The PCR identification method was as follows: plant leaves were taken, genomic DNA was extracted, and a primer pair consisting of bar-F and bar-R was used for PCR amplification. If an amplified product of about 262 bps was obtained, the PCR identification result was positive, and the plant was a transgenic plant. If no amplified product was obtained, the PCR identification result was negative, and the plant was a non-transgenic plant.

bar-F:
GAAGGCACGCAACGCCTACGA;
bar-R:
CCAGAAACCCACGTCATGCCA.

The recombinant plasmid A was used for step two, and three randomly selected transgenic plants were named, namely YT1-1 plant, YT1-2 plant, and YT1-3 plant. The recombinant plasmid B was used to perform step two, and three randomly selected transgenic plants were named, namely YT2-1 plant, YT2-2 plant, and YT2-3 plant. The recombinant plasmid C was used for step two, and three randomly selected transgenic plants were named, namely QT1-1 plant, QT1-2 plant, and QT1-3 plant.

### III. Obtaining Backcrossed Separated Offspring

The T0 generation plants were selfed, the seeds were harvested, and the seeds were then cultivated into plants, that is, the T1 generation plants. The transgenic plants were screened from the T1 generation plants. The PCR identification method was the same as 3 in step II. The T1 generation transgenic plant was used as the female parent, and the corn inbred line Q11 was used as the male parent. The seeds were cultivated into plants, that is, the F1 generation plants. The transgenic plants were screened from the F1 generation plants, and the PCR identification method was the same as 3 in step II. The F1 generation transgenic plant was used as the female parent, and the corn inbred line Q11 was used as the male parent. The seeds were cultivated into plants, that is, the BC₁F₁ generation plants. The transgenic plants and non-transgenic plants were screened from the BC₁F₁ generation plants, and the PCR identification method was the same as 3 in step II. The BC₁F₁ generation transgenic plant was used as the female parent, and the corn inbred line Q11 was used as the male parent. The seeds were cultivated into plants, that is, BC₂F₁ generation plants. The transgenic plants and non-transgenic plants were screened from the BC₂F₁ generation plants, and the PCR identification method was the same as 3 in step II. The BC₂F₁ generation transgenic plant was used as the female parent, and the corn inbred line Q11 was used as the male parent. The seeds were cultivated into plants, that is, BC₃F₁ generation plants. The transgenic plants and non-transgenic plants were screened from the BC₃F₁ generation plants, and the PCR identification method was the same as 3 in step II.

The T0 generation plants were: YT1-1 plant, YT1-2 plant, YT1-3 plant, YT2-1 plant, YT2-2 plant, YT2-3 plant, QT1-1 plant, QT1-2 plant, and QT1-3 plant.

### IV. Identification of Plant Disease Resistance

The tested plants were: BC₃F₁ transgenic plants and non-transgenic plants of YT1-1 plants, BC₃F₁ transgenic plants and non-transgenic plants of YT1-2 plants, BC₃F₁ transgenic plants and non-transgenic plants of YT1-3 plants, BC₁F₁ generation transgenic plants and non-transgenic plants of YT2-1 plants, BC₁F₁ generation transgenic plants and non-transgenic plants of YT2-2 plants, BC₁F₁ generation transgenic plants and non-transgenic plants of YT2-3 plants, BC₂F₁ generation transgenic plants and non-transgenic plants of QT1-1 plants, BC₁F₁ generation transgenic plants and non-transgenic plants of QT1-2 plants, and BC₃F₁ generation transgenic plants and non-transgenic plants of QT1-3 plants.

### 1. Identification of Gene Expression Level

The total RNA from the leaves of the tested plants was extracted and reverse transcribed to obtain the cDNA. With the GAPDH gene as the internal reference gene, the relative expression level of the ZmPK gene was detected by qPCR. The results are shown in FIG. 2.

Primer pair used to detect ZmPK gene:

ZmPK-F:
GCGTTGCCGTCAAGCGCAT;
ZmPK-R:
GCTCCATCACAATGTACACGT.

Primer pair used to detect GAPDH gene:

GAPDH-F:
ATCAACGGCTTCGGAAGGAT;
GAPDH-R:
CCGTGGACGGTGTCGTACTT.

### 2. Identification of Disease Resistance

The disease resistance identification was carried out in Shangzhuang Experimental Base of China Agricultural University.

The pathogen of gray spot disease: Cercospora zeina.

The test plants were cultured under normal conditions, and pathogenic bacteria were inoculated during the bell mouth stage, and then the normal culture was continued. After two weeks of pollination, the phenotypic investigation was carried out, and the disease index (DSI) was calculated in a graded investigation. The specific method of inoculating pathogenic bacteria (bacterial fluid filling method) was as follows: the spores of the gray spot disease-causing fungus were added in sterile water to obtain a spore suspension with a spore concentration of 2-4×10⁵/mL, and a syringe was used to infuse the spore suspension into the corn bell mouth, and 5 ml were infused per corn plant.

The grading standard of the disease level (X represents the percentage of the diseased spot area to the leaf area):

Level 1 (assigned value was 0): X≤5%;

Level 3 (assigned value was 0.25): 5%<X≤10%;

Level 5 (assigned value was 0.5): 10%<X≤30%;

Level 7 (assigned value was 0.75): 30%<X≤70%;

Level 9 (assigned value was 1): 70%<X≤100%.

$$Disease index ( DSI ) ( \% ) = \sum ( assigned value corresponding to the disease level \times the number of plants at this level ) \times 100 \text{/} 1 \times total number of plants$$

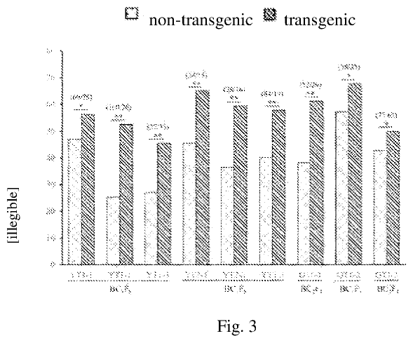

The results are shown in FIG. 3. In FIG. 3, the parentheses indicate the number of plants, the number before the dividing line is the number of non-transgenic plants, and the number after the dividing line is the number of transgenic plants.

The results show that, compared with non-transgenic plants, the expression of ZmPK gene in transgenic plants was significantly higher, and the disease index of corresponding transgenic plants was also significantly higher.

### EXAMPLE 3

### Obtaining and Identifying Plants in Which the Expression is Inhibited

### I. Obtaining Plants in Which the Expression is Inhibited

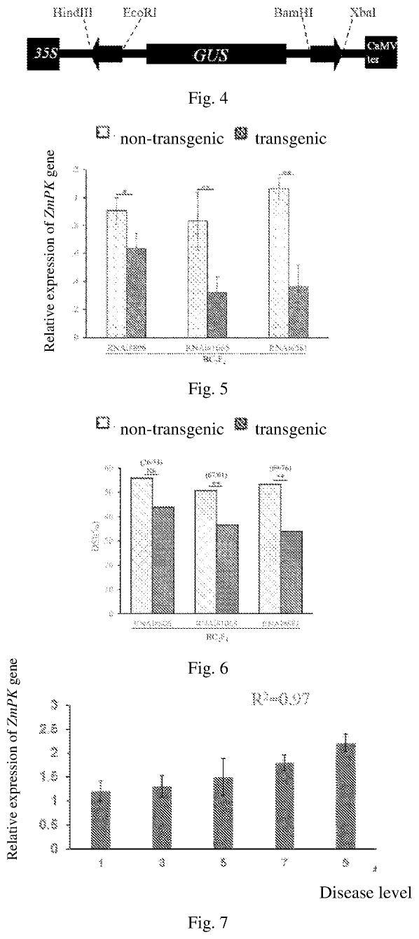

1. The pGreen-HY104 vector was used as the starting vector, the forward fragment was inserted between the BamHI and XbaI restriction sites, and the reverse fragment was inserted between the HindIII and EcoRI restriction sites, so as to obtain an RNAi interference vector. The RNAi interference vector was verified by sequencing. The forward segment and the reverse segment were in a reverse complementary relationship. The forward fragment is shown in Sequence 11 in the sequence listing. The structure diagram of the RNAi interference vector is shown in FIG. 4.

2. The RNAi interference vector obtained in step 1 was introduced into Agrobacterium EHA105 to obtain recombinant Agrobacterium.

3. The recombinant Agrobacterium obtained in step 2 was taken, and the Agrobacterium-mediated method was used to genetically transform the immature embryos of the corn inbred line B73-329 to obtain the T0 generation plants.

4. The T0 generation plants were selfed, the seeds were harvested, and the seeds were then cultivated into plants, that is, the T1 generation plants.

5. The T0 generation plants were identified by PCR.

The PCR identification method was the same as 3 in step II in Example 2.

Three randomly selected transgenic plants were named as RNAi#806 plants, RNAi#1065 plants, and RNAi#581 plants.

### II. Obtaining Backcrossed Separate Population

The PCR identification method was the same as 3 in step II in Example 2.

The T0 generation plants were selfed, the seeds were harvested, and the seeds were then cultivated into plants, that is, the T1 generation plants. The transgenic plants were screened from the T1 generation plants (PCR identification). The T1 generation transgenic plant was used as the female parent, and the corn inbred line Q11 was used as the male parent. The seeds were cultivated into plants, that is, the F1 generation plants. The transgenic plants were screened from the F1 generation plants (PCR identification). The F1 generation transgenic plant was used as the female parent, and the corn inbred line Q11 was used as the male parent. The seeds were cultivated into plants, that is, the BC₁F₁ generation plants. The transgenic plants and non-transgenic plants were screened from the BC₁F₁ generation plants (PCR identification). The BC₁F₁ generation transgenic plant was used as the female parent, and the corn inbred line Q11 was used as the male parent. The seeds were cultivated into plants, that is, BC₂F₁ generation plants. The transgenic plants and non-transgenic plants were screened from the BC₂F₁ generation plants (PCR identification).

The T0 generation plants were: RNAi#806 plant, RNAi#1065 plant, and RNAi#581 plant.

### III. Identification of Plant Disease Resistance

The tested plants were: BC₂F₁ transgenic plants and non-transgenic plants of RNAi#806 plants, BC₂F₁ transgenic plants and non-transgenic plants of RNAi#1065 plants, and BC₂F₁ transgenic plants and non-transgenic plants of RNAi#581 plants.

### 1. Identification of Gene Expression Level

The method was the same as 1 in step IV in Example 2.

The results are shown in FIG. 5.

### 2. Identification of Disease Resistance

The method was the same as 2 in step IV in Example 2.

The results are shown in FIG. 6. In FIG. 6, the parentheses indicate the number of plants, the number before the dividing line is the number of non-transgenic plants, and the number after the dividing line is the number of transgenic plants.

The results show that, compared with non-transgenic plants, the expression of ZmPK gene in transgenic plants was significantly reduced, and the disease index of the corresponding transgenic plants was significantly reduced.

### EXAMPLE 4

### Analysis of the Correlation Between Gene Expression and Disease Resistance

The original parents of the positioning population were corn inbred line Y32 and corn inbred line Q11. Through continuous crossing and backcrossing, a high-generation backcrossing segregation population was constructed.

Three plants with disease levels of 1, 3, 5, 7, and 9 in the positioning population were randomly selected, and gene expression level identification (the method was the same as that of 1 in step IV in Example 2) and disease resistance identification (the method was the same as 2 in step IV in Example 2) were performed. The correlation between the expression of ZmPK gene and the level of gray leaf spot was calculated. The results are shown in FIG. 7. There was a significant positive correlation between the expression of ZmPK gene and the level of gray leaf spot. This further confirms the negative correlation between ZmPK gene expression and plant resistance to gray leaf spot.

### EXAMPLE 5

### Obtaining and Identifying Complementary Transgenic Plants

### I. Construction of Recombinant Expression Vector

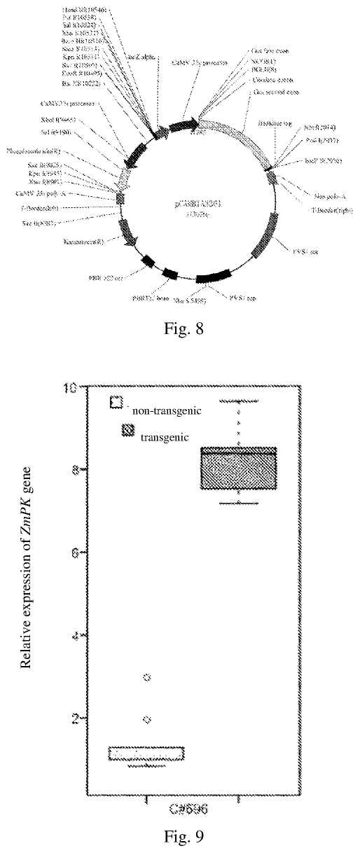

A recombinant plasmid was prepared. According to the sequencing results, the structure of the recombinant plasmid was described as follows: the DNA molecule shown in Sequence 12 in the sequence list was inserted into the BamHI restriction site of the pCAMBIA3301 vector. FIG. 8 shows a schematic diagram of the structure of the pCAMBIA3301 vector.

### II. Obtaining Complementary Transgenic Plants

1. The recombinant plasmid prepared in step I was introduced into Agrobacterium EHA105 to obtain recombinant Agrobacterium.

2. The recombinant Agrobacterium obtained in step 1 was taken, and the Agrobacterium-mediated method was used to genetically transform the immature embryos of the corn inbred line B73 to obtain the T0 generation plants.

3. The T0 generation plants were identified by PCR.

The PCR identification method was the same as 3 in step II in Example 2.

A randomly selected transgenic plant was named C#596 plant.

### III. Obtaining Backcrossed Separated Offspring

The T0 generation plants were selfed, the seeds were harvested, and the seeds were then cultivated into plants, that is, the T1 generation plants. The transgenic plants were screened from the T1 generation plants. The PCR identification method was the same as 3 in step II. The T1 generation transgenic plant was used as the female parent, and the corn inbred line Q11 was used as the male parent. The seeds were cultivated into plants, that is, the F1 generation plants. The transgenic plants were screened from the F1 generation plants, and the PCR identification method was the same as 3 in step II. The F1 generation transgenic plant was used as the female parent, and the corn inbred line Q11 was used as the male parent. The seeds were cultivated into plants, that is, the BC₁F₁ generation plants.

### IV. Identification of Plant Disease Resistance

The tested plants were: BC₁F₁ generation transgenic plants and non-transgenic plants of C#595 plants, and BC₁F₁ generation transgenic plants and non-transgenic plants of C#596 plants.

### 1. Identification of Gene Expression Level

The method was the same as 1 in step IV in Example 2.

The results are shown in FIG. 9.

### 2. Identification of Disease Resistance

The method was the same as 2 in step IV in Example 2.

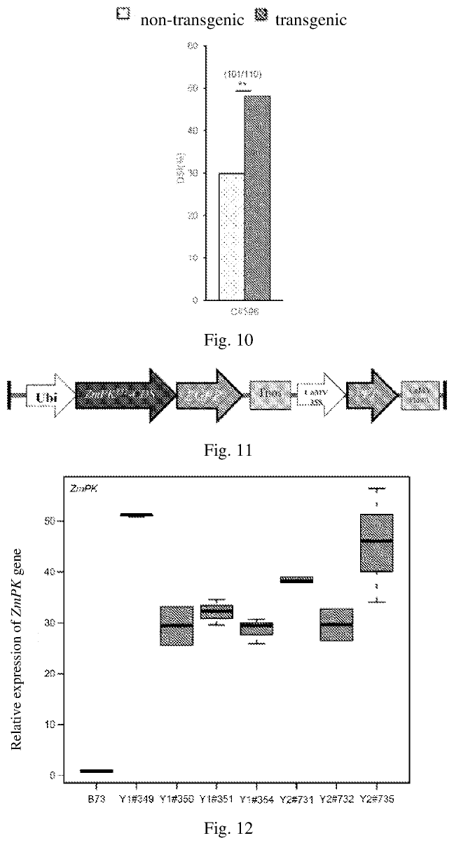

The results are shown in FIG. 10. In FIG. 10, the parentheses indicate the number of plants, the number before the dividing line is the number of non-transgenic plants, and the number after the dividing line is the number of transgenic plants.

The results show that, compared with non-transgenic plants, the expression of ZmPK gene in transgenic plants was significantly higher, and the disease index of corresponding transgenic plants was also significantly higher.

### EXAMPLE 6

### Obtaining and Identifying Pure Lines of Fusion Overexpression Plants

### 1. Construction of Recombinant Expression Vector

The recombinant plasmid E and recombinant plasmid F were prepared respectively. According to the sequencing results, the structure of recombinant plasmid E was described as follows: at the XcmI restriction site of the pBCXUN vector, a DNA molecule shown in Sequence 13 of the sequence listing was inserted. According to the sequencing results, the structure of recombinant plasmid F was described as follows: at the XcmI restriction site of the pBCXUN vector, a DNA molecule shown in Sequence 14 of the sequence listing was inserted. The schematic diagram of the structure of recombinant plasmid E and recombinant plasmid F is shown in FIG. 11.

### II. Obtaining Pure Lines of Fusion Overexpression Plants

1. The recombinant plasmid was introduced into Agrobacterium EHA105 to obtain recombinant Agrobacterium.

2. The recombinant Agrobacterium obtained in step 1 was taken, and the Agrobacterium-mediated method was used to genetically transform the immature embryos of the corn inbred line B73 to obtain T0 generation plants.

3. The T0 generation plants were identified by PCR.

The PCR identification method was the same as 3 in step II of Example 2.

The recombinant plasmid E was used to carry out step II, and four randomly selected transgenic plants were named: Y1#349 plant, Y1#350 plant, Y1#351 plant, and Y1#354 plant. The recombinant plasmid F was used to carry out step II, and three randomly selected transgenic plants were named, namely Y2#731 plant, Y2#732 plant, and Y2#735 plant.

4. The T0 generation plants were selfed and the seeds were harvested. The seeds were cultivated into plants, which were the T1 generation plants. The transgenic plants (identified by PCR) were screened from the T1 generation plants for selfing, and the seeds were harvested. The T2 generation plants were cultivated from the seeds into the plants. The transgenic plants (identified by PCR) from the T2 generation plants were screened for selfing, and the seeds were harvested. The seeds were cultivated to plants, that is, the T3 generation plants. The PCR identification method was the same as 3 in step II in Example 2. For a T2 generation plant, if the T3 generation plants obtained by selfing were all transgenic plants, the T2 generation plant was a homozygous transgenic plant. The offspring obtained by selfing of the homozygous transgenic plant was a homozygous transgenic line.

The following homozygous transgenic strains were obtained: Y1#349 strain, Y1#350 strain, Y1#351 strain, Y1#354 strain, Y2#731 strain, Y2#732 strain, and Y2#735 strain.

### III. Identification of Plant Disease Resistance

The tested plants were: T3 generation plants of Y1#349 line, T3 generation plants of Y1#350 line, T3 generation plants of Y1#351 line, T3 generation plants of Y1#354 line, T3 generation plants of Y2#731 line, T3 generation plants of Y2#732 line, T3 generation plants of Y2#735 line, and the corn inbred line B73 plants.

### 1. Identification of Gene Expression Level

The method was the same as 1 in step IV in Example 2.

The results are shown in FIG. 12.

### 2. Identification of Disease Resistance

The method was the same as 2 in Step IV in Example 2.

The results are shown in FIG. 13. In FIG. 13, the parentheses indicate the number of plants.

The results show that compared with the corn inbred line B73 plants (control non-transgenic plants), the expression of ZmPK gene in the transgenic plants was significantly higher, and the disease index of the corresponding transgenic plants was also significantly higher.

### Example 7

### Obtaining and Identifying Gene-Edited Plants

### 1. Construction of Gene Editing Vector

A gene editing vector was prepared, that is, a recombinant plasmid was obtained by: inserting the double-stranded DNA molecule shown in Sequence 16 in the sequence listing into the BsaI restriction site of the pBUE411 vector. The recombinant plasmid was verified by sequencing. The gene editing vector encoded two sgRNAs, the target sequence binding region of one sgRNA is shown in Sequence 17 in the sequence listing, and the target sequence binding region of the other sgRNA is shown in Sequence 18 in the sequence listing. The gene editing vector contained the Cas9 gene and expressed the Cas9 protein.

### II. Obtaining Gene-Edited Plants

1. The gene editing vector was introduced into Agrobacterium EHA105 to obtain recombinant Agrobacterium.

2. The recombinant Agrobacterium obtained in step 1 was taken, and the Agrobacterium-mediated method was used to genetically transform the immature embryos of the corn inbred line B73 to obtain T0 generation plants.

3. The plants with mutations in the target sequence were screened from the T0 generation plants.

The specific method was as follows: the plant leaves were taken, the genomic DNA was extracted, a primer pair composed of F and R was used for PCR amplification, and the PCR amplification product was recovered and sequenced. The sequencing result was compared with the wild-type sequence (the wild-type sequence is shown in the 9599-10985 in Sequence 12 in the sequence listing), and plants with different sequences were screened.

F:
5′-TTGAGGTCATTGTCTCAGCC-3′;
R:
5′-AGCAGCTTGGCAGCGTATG-3′.

4. The T0 generation plants screened in step 3 were taken, and used for selfing and then harvested to obtain the seeds. The seeds were cultivated into plants, which were the T1 generation plants.

5. The gene-edited plants were screened from T1 generation plants.

(1) The plants were subjected to PCR identification with the bar gene in the gene editing vector as the target. PCR identification method was as follows: plant leaves were taken, genomic DNA was extracted, and the primer pair containing bar-F and bar-R was used for PCR amplification. If no amplified product was obtained, the PCR identification result was negative.

(2) The plants with the target region of gene editing were identified as the target. The identification method was the same as step 3.

If a plant is identified as negative by PCR in step (1), and the identification result in step (2) shows that it is different from the wild-type sequence and is homozygous (and the two chromosomes are identical), the plant is a gene-edited plant.

Two gene-edited plants were obtained, named ZmPK-KO#1 plant and ZmPK-KO#2 plant, respectively.

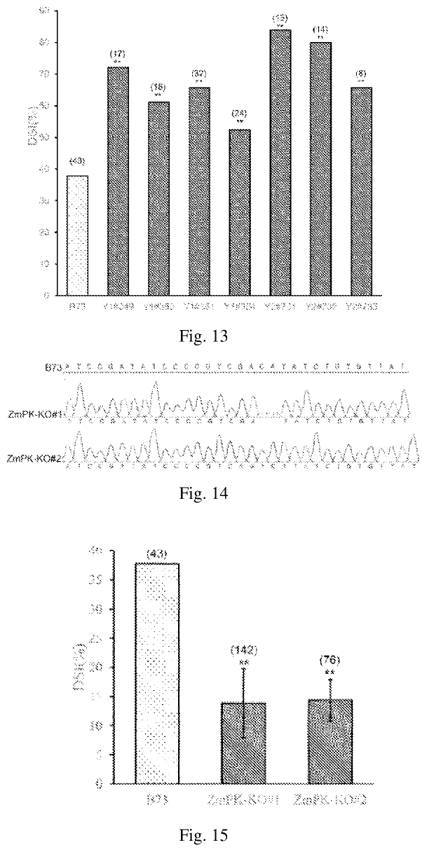

The sequencing results of step (2) of the two gene editing plants are shown in FIG. 14. Compared with B73, two nucleotides were missing in the ZmPK-KO#1 plant, and a nucleotide was inserted into the ZmPK-KO#2 plant. Both caused frameshift mutations.

6. The gene-edited plants were taken, self-bred, and offspring plants were obtained, that is, the gene-edited plants.

Two gene editing lines were obtained, and named ZmPK-KO#1 strain and ZmPK-KO#2 strain, respectively.

### 3. Identification of Disease Resistance of Gene-Edited Plants

The tested plants were: T2 generation plants of ZmPK-KO#1 line, T2 generation plants of ZmPK-KO#2 line, and corn inbred line B73 plants.

The method was the same as 2 in step IV in Example 2.

The results are shown in FIG. 15. In FIG. 15, the parentheses indicate the number of plants.

Compared with corn inbred line B73 plants, the disease index of gene-edited plants was significantly reduced.

### INDUSTRIAL APPLICABILITY

The present invention provides a major gene ZmPK for resistance to gray leaf spot of corn. Through transgene complementation, overexpression, CRISPR knockout and RNAi interference experiments, the gene's anti-grey leaf spot function-negative regulation of gray leaf spot resistance has been proved. The present invention has great application value for the breeding of corn against gray leaf spot.

1. A protein, comprising:
(a1) an amino acid of SEQ ID NO: 2; or
(a2) an amino acid of SEQ ID NO: 4; or
(a3) an amino acid of SEQ ID NO: 7; or
(a4) an amino acid of SEQ ID NO: 9; or
(a5) an amino acid of SEQ ID NO: 1 or SEQ ID NO: 7 further comprising a peptide a tag linked to an N-terminus or/and a C-terminus of SEQ ID NO: 2, SEQ ID NO: 4, SEQ ID NO: 7 or SEQ ID NO: 9; or
(a6) an amino acid comprising the following three segments from N-terminus to C-terminus: the amino acid of any one of (a1) to (a4), a connecting peptide, and an EGFP protein; or
(a7) an amino acid that differs from SEQ ID NO: 2, SEQ ID NO: 4, SEQ ID NO: 7 or SEQ ID NO: 9 by substituting and/or deleting and/or adding one of the amino acid residues of the respective sequence of SEQ ID NO: 2, SEQ ID NO: 4, SEQ ID NO: 7 or SEQ ID NO: 9; or
(a8) an amino acid having at least 90% sequence identity with SEQ ID NO: 2, SEQ ID NO: 4, SEQ ID NO: 7 or SEQ ID NO: 9.

2. A nucleic acid molecule, which encodes the protein according to claim 1.

3. The nucleic acid molecule according to claim 2, wherein the nucleic acid molecule comprises:
(b1) nucleotides 56 to 1618 [[in]] of SEQ ID NO: 3 in the sequence listing; or
(b2) SEQ ID NO: 3; or
(b3) nucleotides 56 to 1624 of SEQ ID NO: 5; or
(b4) SEQ ID NO: 5; or
(b5) SEQ ID NO: 1; or
(b6) nucleotides 56 to 1618 of SEQ ID NO: 8; or
(b7) SEQ ID NO: 8; or
(b8) nucleotides 56 to 1624 of SEQ ID NO: 10; or
(b9) SEQ ID NO: 10; or
(b10) SEQ ID NO: 6; or
(b11) SEQ ID NO: 12; or
(b12) SEQ ID NO: 13; or
(b13) SEQ ID NO: 14; or
(b14) an amino acid having at least 90% sequence identity with SEQ ID NO: 1, SEQ ID NO: 3, SEQ ID NO: 5, SEQ ID NO: 6, SEQ ID NO: 8, SEQ ID NO: 10, SEQ ID NO: 12, SEQ ID NO: 13, or SEQ ID NO: 14; or
(b15) a DNA molecule that hybridizes to any one of (b1) to (b13) under a stringent condition, and encodes the protein.

4. An expression cassette, a recombinant vector or a recombinant microorganism comprising the nucleic acid molecule according to claim 2.

5. A method of reducing the resistance of a plant to a disease caused by Cercospora zeina, optionally wherein the disease is gray leaf spot disease, said method comprising expressing the protein according to claim 1 in said plant.

6. The method according to claim 5, wherein the plant is a plant of the genus Zea.

7. The method according to claim 5 wherein said plant is transformed with a nucleic acid encoding an amino acid having at least 90% sequence identity with SEQ ID NO: 2, SEQ ID NO: 4, SEQ ID NO: 7 or SEQ ID NO: 9 to produce a transgenic plant with altered resistance to gray leaf spot.

8. The method according to claim 7, wherein the plant is a plant of the genus Zea.

9. A method for inhibiting an activity of the protein according to claim 1 in a plant and/or for reducing an abundance of the protein according to claim 1 in a plant to enhance the disease resistance of the plant to gray leaf spot, said method comprising introducing an inhibitor into the cells of said plant, wherein said inhibitor interferes with the activity of the protein according to claim 1.

10. The application according to claim 9, wherein the plant is a plant of the genus Zea.

11. A method for inhibiting transcription of the nucleic acid molecule according to claim 3 and/or for inhibiting an expression of the nucleic acid molecule according to claim 3 and/or for gene editing of the nucleic acid molecule according to claim 3 to enhance the disease resistance of the plant to gray leaf spot, said method comprising introducing an interference vector into the cells of said plant, wherein said interference vector encodes iRNA that inhibits the expression of the nucleic acid molecule according to claim 3.

12. The method according to claim 11, wherein the plant is a plant of the genus Zea.

13. A method for preparing a transgenic plant, comprising the steps of: introducing the nucleic acid molecule according to claim 3 into a starting plant to obtain a transgenic plant with reduced gray leaf spot resistance.

14. The method according to claim 13, wherein the starting plant is a plant of the genus Zea.

15. A plant breeding method, comprising the following steps:
increasing a content and/or activity of the protein according to claim 1 in a target plant, thereby reducing the gray leaf spot resistance of the target plant.

16. The method according to claim 15, wherein the target plant is a plant of the genus Zea.

17. A method for preparing a transgenic plant, comprising the following steps: inhibiting an expression of the nucleic acid molecule according to claim 3 in a starting plant to obtain a transgenic plant with increased gray leaf spot resistance.

18. The method according to claim 17, wherein the starting plant is a plant of the genus Zea.

19. A plant breeding method, comprising the following steps:
reducing a content and/or activity of the protein according to claim 1 in a target plant, thereby increasing the disease resistance of the target plant to gray leaf spot.

20. The method according to claim 19, wherein the target plant is a plant of the genus Zea.

21. A plant breeding method, comprising the steps of:
performing gene editing on a specific gene in a genome of a starting plant to increase the gray leaf spot resistance of the target plant; wherein said specific gene encodes the protein according to claim 1.

22. The method according to claim 21, wherein the starting plant is a plant of the genus Zea.
